(19) United States
(12) Patent Application Publication
(10) Pub. No.: US 2008/0124792 A1
(43) Pub. Date: May 29, 2008

(54) Hybrid promoters

(75) Inventor: Alex J. Harvey, Athens, GA (US)

Correspondence Address: AVIGENICS, INC., 111 RIVERBEND ROAD, ATHENS, GA, 30605, US

(73) Assignee: AviGenics, Inc.

(21) Appl. No.: 11/880,838

(22) Filed: Jul. 24, 2007

Related U.S. Application Data
(60) Provisional application No. 60/836,098, filed on Aug. 7, 2006.

(51) Int. Cl. A01K 67/033; C07H 21/04; C12N 15/00; C12P 21/00

(52) U.S. Cl. 435/320.1; 536/24.1; 800/19; 800/4

(57) ABSTRACT

The invention includes hybrid promoters containing an LTR component and a promoter component, transgenic avians containing the hybrid promoters in their genome and methods of making the transgenic avians.

### RELATED APPLICATION INFORMATION

This application claims priority to U.S. provisional patent application No. 60/836,098, filed Aug. 7, 2006, the disclosure of which is incorporated in its entirety herein by reference.

### BACKGROUND

The present invention relates generally to recombinant gene expression controlling regions, i.e., hybrid promoters. The invention includes recombinant nucleic acid molecules and expression vectors, transfected cells and transgenic animals such as avians that include hybrid promoters operably linked to a nucleic acid of interest.

The field of transgenics was initially developed to understand the action of a single gene in the context of the whole animal and the phenomena of gene activation, expression, and interaction. Transgenics has also been used to produce models for various diseases in humans and other animals and is a useful tool for the study of genetics, and the understanding of genetic mechanisms and function. From an economic perspective, the use of transgenic technology for the production of specific proteins such as substances of pharmaceutical interest (Gordon et al., (1987) Biotechnology 5: 1183-1187; Wilmut et al., (1990) Theriogenology 33: 113-123) offers significant advantages over more conventional methods of protein production by gene expression.

Heterologous nucleic acids have been engineered so that an expressed protein may be joined to a protein or peptide that will allow secretion of the transgenic expression product into milk or urine, from which the protein may then be recovered. These procedures have had limited success and may require maintenance of herds of large species, such as cows, sheep, or goats. Such animals typically have exceedingly long developmental periods and are costly to maintain.

One alternative that has shown great usefulness for heterologous gene expression is the avian reproductive system. The production of an avian egg begins with formation of a large yolk in the ovary of the hen. The unfertilized oocyte or ovum is positioned on top of the yolk sac. After ovulation, the yolk and ovum pass into the infundibulum of the oviduct where it is fertilized, if sperm are present, and then moves into the magnum of the oviduct which is lined with tubular gland cells. Tubular gland cells secrete the egg-white proteins, including ovalbumin, ovomucoid, ovoinhibitor, conalbumin, ovomucin and lysozyme, into the lumen of the magnum where they are deposited onto the yolk and ovum.

The hen oviduct has been shown to be an excellent protein bioreactor because of the high levels of protein production, the promise of proper folding and post-translation modification of the target protein, the ease of product recovery, and the shorter developmental period of chickens compared to other animal species used for heterologous gene expression. As a result, efforts have been made to produce transgenic chickens expressing increased amounts of exogenous protein in the oviduct.

Producing avians that can routinely provide for a higher level of protein production in the oviduct has been a goal in the field of avian transgenesis since the first transgenic birds which laid eggs containing exogenous proteins were disclosed. See U.S. Pat. No. 6,730,822, issued May 4, 2004, the disclosure of which is incorporated in its entirety herein by reference. What is needed are promoters that function to produce greater quantities of exogenous protein in the avian oviduct for packaging into eggs laid by transgenic birds.

### SUMMARY

It has been discovered that fusing an LTR component to a promoter component can produce a promoter with high transcriptional activity. In one embodiment, the invention is drawn to hybrid promoters and methods of using the hybrid promoters wherein the hybrid promoters comprise a retroviral LTR (long terminal repeat) component linked to a constitutive promoter component. In a particularly useful embodiment of the invention, the 3′ end of the LTR component is linked to the 5′ end of the promoter component to form a hybrid promoter. The term LTR component as used herein refers to a retroviral LTR or a portion of a retroviral LTR. The term promoter component as used herein refers to a constitutive promoter or a portion of a constitutive promoter.

In one aspect, the invention is directed to a transgenic avian containing a hybrid promoter comprising an LTR component and a promoter component. In particular, the hybrid promoter is present in the genome of the avian and may be present in one of the avian's natural chromosomes. The transgenic avian of the invention may be, without limitation, a chicken, turkey, duck, goose, quail, pheasant, parrot, finche, hawk, crow and ratite including ostrich, emu and cassowary.

In one particularly useful embodiment, a tubular gland cell of an avian produced in accordance with the invention contains the hybrid promoter.

The invention also includes methods of making avians that contain a hybrid promoter in their genome. The methods may include producing a transgenic avian containing in its genome a hybrid promoter comprising an LTR component and a promoter component wherein the hybrid promoter is operably linked to a protein coding sequence and the protein is produced in the avian and is packaged into a hard shell egg laid by the avian. In one embodiment, the protein is not normally produced in an avian. In one particularly useful embodiment, the protein is a therapeutic protein such as a protein (i.e., an amino acid sequence) which is normally produced in a human.

In one aspect, a hybrid promoter of the invention contains an LTR component and a promoter component, the promoter component being 3′ of the LTR component. That is, the LTR component and the promoter component are both present on the same DNA molecule and the LTR component is 5′ of the promoter component where the directionality is determined by the position of a coding sequence present on the same DNA molecule as the hybrid promoter and operably linked to the hybrid promoter wherein the coding sequence is 3′ of the hybrid promoter.

In one aspect, the 3′ end of the LTR component is linked to the 5′ end of the promoter component to produce a hybrid promoter of the invention. In one particularly useful embodiment, the hybrid promoter is operably linked to a nucleotide coding sequence which may encode a protein.

In one aspect, the hybrid promoter facilitates transcription of a coding sequence operably linked to the hybrid promoter by an amount greater than that of the promoter component. In another aspect, the hybrid promoter facilitates transcription of a coding sequence by an amount greater than that of the LTR component.

In one aspect, the hybrid promoter is contained in a vector. For example, the hybrid promoter may be contained in a retroviral vector. In one embodiment, the hybrid promoters of the invention are contained in a SIN retroviral vector.

In one embodiment, the hybrid promoter is contained in a retroviral vector and the LTR component of the hybrid promoter serves as the 5′ LTR of the retroviral vector. For example, the LTR component can serve as the 5′ LTR of the retroviral vector during the integration process of the vector.

In one aspect, the LTR component of the hybrid promoters of the invention contains an LTR of one of the following retroviruses or a portion of an LTR of one of the following retroviruses: Rous Sarcoma Virus (RSV), Murine Leukemia Virus (MLV), Molony Murine Sarcoma Virus (MMSV), Moloney Murine Leukemia Virus (MMLV), Avian Leokosis Virus (ALV) and lentivirus, e.g., human immunodeficiency virus (HIV), feline immunodeficiency virus (FIV), bovine immunodeficiency virus (BIV) and simian immunodeficiency virus (SIV).

In one aspect, the promoter component of the hybrid promoters of the invention contains one of the following promoters or contains a portion of one of the following promoters: cytomegalovirus (CMV) promoter, ef-1a promoter, PGK promoter (phosphoglycerate kinase) and beta actin promoter, e.g., chicken beta actin promoter, human beta actin promoter and bovine beta actin promoter.

The invention contemplates the production of any useful protein in accordance with the invention including pharmaceutical or therapeutic proteins, including, but not limited to those disclosed herein. In a particularly useful embodiment, the invention is drawn to the production of human proteins. For example, the invention encompasses the production of human antibodies. The invention also contemplates the production of human cytokines such as G-CSF, GM-CSF, EPO, interferon, including, without limitation, interferon alpha, e.g., interferon alpha 2, interferon beta, e.g., interferon beta 1, and interferon gamma.

One important aspect of the present invention relates to avian hard shell eggs (e.g., chicken hard shell eggs) which contain an exogenous peptide or protein including, but not limited to, a pharmaceutical or therapeutic protein. The exogenous peptide or protein may be encoded by a transgene of a transgenic avian. In one embodiment, the exogenous peptide or protein (e.g., pharmaceutical protein) is glycosylated.

The protein encoded by the transgene of the transgenic avian may be present in certain amounts in eggs laid by the transgenic avian. In one embodiment, the protein is present in an amount in a range of between about 0.01 ug per hard-shell egg and about 1 gram per hard-shell egg. In another embodiment, the protein is present in an amount in a range of between about 1 ug per hard-shell egg and about 1 gram per hard-shell egg. For example, the protein may be present in an amount in a range of between about 10 ug per hard-shell egg and about 1 gram per hard-shell egg (e.g., a range of between about 10 ug per hard-shell egg and about 400 milligrams per hard-shell egg).

In one embodiment, the exogenous protein (e.g., exogenous pharmaceutical or therapeutic protein) produced in a transgenic avian such as a G1 transgenic avian is present in the egg white of the egg laid by the transgenic avian. In one embodiment, the protein is present in an amount in a range of between about 1 ng per milliliter of egg white and about 0.2 gram per milliliter of egg white. For example, the protein may be present in an amount in a range of between about 0.1 ug per milliliter of egg white and about 0.2 gram per milliliter of egg white (e.g., the protein may be present in an amount in a range of between about 1 ug per milliliter of egg white and about 100 milligrams per milliliter of egg white. In one embodiment, the protein is present in an amount in a range of between about 1 ug per milliliter of egg white and about 50 milligrams per milliliter of egg white. For example, the protein may be present in an amount in a range of about 1 ug per milliliter of egg white and about 10 milligrams per milliliter of egg white (e.g., the protein may be present in an amount in a range of between about 1 ug per milliliter of egg white and about 1 milligrams per milliliter of egg white).

Expression levels in the eggs of G1 hens produced with conventional promoters such as an intact CMV promoter can be typically 7 ug/ml of egg white or approximately 200 ug per egg.

Any combination of features described herein is included within the scope of the present invention provided that the features included in any such combination are not mutually inconsistent. Such combinations will be apparent based on this specification and on the knowledge of one of ordinary skill in the art.

Additional objects and aspects of the present invention will become more apparent upon review of the description of the figures, definitions, abbreviations and detailed description set forth below when taken in conjunction with the accompanying figures, which are briefly described as follows.

### DESCRIPTION OF THE FIGURES

FIG. 1A shows a fragment of pNLB-CMV-Des-Arg166-EPO. FIG. 1B (SEQ ID NO: 1) shows the nucleotide sequence of FIG. 1A. The approximate coordinates of specific components of the vector as shown in FIG. 1B are:
enhancer region Start: 1 End: 98
CMV enhancer Start: 2589 End: 2995
LTR(RAV2) Start: 1 End: 346
U3 Start: 1 End: 245
R Start: 246 End: 266
region deleted in EP407 Start: 262 End: 2822
U5 Start: 267 End: 346
CMV Promoter Start: 2489 End: 3169
+1 site Start: 3113 End: 3113
+1 site Start: 246 End: 246
TATA Start: 216 End: 222
TATA Start: 3084 End: 3090

FIG. 2A shows the provirus region of pNLB-407P-hG-CSF containing the hybrid 407-Promoter operably linked to a coding sequence of a protein of interest, in this case G-CSF. FIG. 2B (SEQ ID NO: 2) shows the nucleotide sequence of FIG. 2A. The approximate coordinates of specific components of the vector as shown in FIG. 2B are:
CAAT signal Start: 2783 End: 2787
Gag Start: 644 End: 899
Neo Start: 912 End: 1707
CDS Start: 3004 End: 3525
Envelope Start: 3917 End: 4294
LTR(RAV2) Start: 1 End: 346
LTR fragment Start: 2294 End: 2554
LTR(RAV2) Start: 4630 End: 4975
AEV1 region Start: 2069 End: 2283
CMV fragment enhancer Start: 2555 End: 2727
CMV fragment promoter Start: 2555 End: 2903
polyA signal Start: 2532 End: 2537
TATA signal Start: 2816 End: 2819

FIG. 3A shows the structure of the pNLB-CMV-Des-Arg166-EPO transgene integrated in the EP407 G1 hen containing the 407-Promoter. FIG. 3B (SEQ ID NO: 3) shows the nucleotide sequences of FIG. 3A. The approximate coordinates of specific components of the transgene as shown in FIG. 3B are:
CAAT signal Start: 490 End: 494
CDS Start: 619 End: 1194
Envelope Start: 1586 End: 1963
Enhancer Start: 262 End: 434
LTR(RAV2) Start: 1 End: 261
LTR (RAV2) Start: 2299 End: 2644
5′ region of 407-Promoter Start: 53 End: 261
3′ region of 407-Promoter Start: 262 End: 1249
polyA signal Start: 239 End: 244
polyA signal Start: 2537 End: 2542
CMV Promoter Start: 262 End: 608
TATA Start: 523 End: 526

FIG. 4 shows the transduction vector pNLB-CMV-Des-Arg166-EPO.

FIG. 5 shows the transduction vector pNLB-CMV-hG-CSF.

FIG. 6 shows the transduction vector pNLB-407-hG-CSF.

FIG. 7 shows a RSV LTR/chicken beta actin promoter hybrid promoter (SEQ ID NO: 4).

FIG. 8 shows a RSV LTR/SV40 promoter hybrid promoter (SEQ ID NO: 5).

FIG. 9 shows a RSV LTR/PGK promoter hybrid promoter (SEQ ID NO: 6).

FIG. 10 shows a RSV LTR/mouse EF1 alpha promoter hybrid promoter (SEQ ID NO: 7).

FIG. 11 shows a HIV LTR/CMV promoter hybrid promoter (SEQ ID NO: 8).

FIG. 12 shows a HIV LTR/Chicken beta actin promoter hybrid promoter (SEQ ID NO: 9).

FIG. 13 shows a HIV LTR/SV40 promoter hybrid promoter (SEQ ID NO: 10).

FIG. 14 shows a HIV LTR/PGK promoter hybrid promoter (SEQ ID NO: 11).

FIG. 15 shows a HIV LTR/mouse EF1 alpha promoter hybrid promoter (SEQ ID NO: 12).

FIG. 16 shows a MMLV LTR/CMV promoter hybrid promoter (SEQ ID NO: 13).

FIG. 17 shows a MMLV LTR/chicken beta actin promoter hybrid promoter (SEQ ID NO: 14).

FIG. 18 shows a MMLV LTR/SV40 promoter hybrid promoter (SEQ ID NO: 15).

FIG. 19 shows a MMLV LTR/PGK promoter hybrid promoter (SEQ ID NO: 16).

FIG. 20 shows a MMLV LTR/mouse EF1 alpha promoter hybrid promoter (SEQ ID NO: 17).

FIG. 21A shows a portion of the pSIN-407-hG-CSF vector LTR to LTR. The complete pSIN-407-hG-CSF vector sequence is shown in SEQ ID NO: 18. The 3′ long terminal repeat (LTR) is self-inactivating (SIN). FIG. 21B shows a portion of the pTombak hG-CSF vector LTR to LTR. The complete pTombak hG-CSF vector is shown in SEQ ID NO: 19. Essentially the R region from about bp 17 to bp 21 (the R region is approximately 21 bp in length) and the U5 region of the 5′ LTR are replaced with the partial CMV promoter to produce the LTR component-CMV component hybrid 407-Promoter of the Tombak vector.

FIG. 22 shows a possible mechanism of operation for reverse transcription of the Tombak vector to produce a DNA molecule capable of integration into the chicken genome; however, it is noted here that the invention is not limited to any particular theory of mechanisms of operation. PBS=Primer binding site; CMV=CMV promoter component; PPT=Polypurine tract.

FIG. 23 shows the vector pAVIJCR-A395.22.3.1-KM which is also shown in SEQ ID NO: 28.

### DEFINITIONS

Definitions of certain terms used in the application are set forth below.

As used herein the terms “amino acid sequence” and “protein” refer to a polymer of amino acids of three or more amino acids in a serial array, linked through peptide bonds. The term “amino acid sequence” includes proteins, protein fragments, protein analogues, oligopeptides and the like. The term amino acid sequence as used herein can also refer to a peptide. The term “amino acid sequences” contemplates amino acid sequences as defined above that are encoded by nucleic acids, produced through recombinant technology (isolated from an appropriate source such as a bird), or synthesized. The term “amino acid sequences” further contemplates amino acid sequences as defined above that include chemically modified amino acids or amino acids covalently or noncovalently linked to labeling ligands.

The term “animal” is used herein to include all vertebrate and invertebrate animals, including humans. It also includes an individual animal in all stages of development, including embryonic and fetal stages.

The term “avian” as used herein refers to any species, subspecies or race of organism of the taxonomic class ava, such as, but not limited to, chicken, turkey, duck, goose, quail, pheasants, parrots, finches, hawks, crows and ratites including ostrich, emu and cassowary. The term includes the various known strains of Gallus gallus, or chickens, (for example, White Leghorn, Brown Leghorn, Barred-Rock, Sussex, New Hampshire, Rhode Island, Ausstralorp, Minorca, Amrox, California Gray, Italian Partidge-colored), as well as other poultry bred for commercial purposes.

The term “antibody” as used herein refers to polyclonal and monoclonal antibodies and fragments thereof, and immunologic binding equivalents thereof. The term “antibody” refers to a homogeneous molecular entity, or a mixture such as a polyclonal serum product made up of a plurality of different molecular entities, and may further comprise any modified or derivatised variant thereof that retains the ability to specifically bind an epitope. A monoclonal antibody is capable of selectively binding to a target antigen or epitope. Antibodies may include, but are not limited to polyclonal antibodies, monoclonal antibodies (mAbs), humanized or chimeric antibodies, camelized antibodies, single chain antibodies (scFvs), Fab fragments, F(ab′)₂ fragments, disulfide-linked Fvs (sdFv) fragments, e.g., as produced by a Fab expression library, anti-idiotypic (anti-Id) antibodies, intrabodies, synthetic antibodies, and epitope-binding fragments of any of the above.

The term “cytokine” as used herein refers to any secreted amino acid sequence that affects the functions of cells and is a molecule that modulates interactions between cells in the immune, inflammatory or hematopoietic responses. A cytokine includes, but is not limited to, monokines and lymphokines regardless of which cells produce them. For instance, a monokine is generally referred to as being produced and secreted by a mononuclear cell, such as a macrophage and/or monocyte. Many other cells however also produce monokines, such as natural killer cells, fibroblasts, basophils, neutrophils, endothelial cells, brain astrocytes, bone marrow stromal cells, epideral keratinocytes and B-lymphocytes. Lymphokines are generally referred to as being produced by lymphocyte cells. Examples of cytokines include, but are not limited to, Interleukin-1 (IL-1), Interleukin-6 (IL-6), Interleukin-8 (IL-8), Tumor Necrosis Factor-alpha (TNF-alpha) and Tumor Necrosis Factor beta (TNF-beta).

The term “coding region” or “coding sequence” as used herein refers to a continuous linear arrangement of nucleotides which can be transcribed to produce RNA. Thereafter, the RNA may be translated into an amino acid sequence. A coding region may include any leader protein sequence or any other region of the protein that may be excised from the translated protein.

The term “complementary” as used herein refers to two nucleic acid molecules that can form specific interactions with one another. In the specific interactions, an adenine base within one strand of a nucleic acid can form two hydrogen bonds with thymine within a second nucleic acid strand when the two nucleic acid strands are in opposing polarities. Also in the specific interactions, a guanine base within one strand of a nucleic acid can form three hydrogen bonds with cytosine within a second nucleic acid strand when the two nucleic acid strands are in opposing polarities. Complementary nucleic acids as referred to herein, may further comprise modified bases wherein a modified adenine may form hydrogen bonds with a thymine or modified thymine, and a modified cytosine may form hydrogen bonds with a guanine or a modified guanine.

The term “expressed” or “expression” as used herein refers to transcription of a coding sequence to yield an RNA nucleic acid molecule at least complementary in part to a region of one of the two nucleic acid strands of a coding sequence. The term “expressed” or “expression” as used herein can also refer to the translation from an RNA nucleic acid molecule producing a protein, an amino acid sequence or a portion thereof.

The term “expression vector” as used herein refers to a nucleic acid vector that comprises an expression controlling region operably linked to a nucleotide sequence encoding at least one amino acid sequence.

As used herein, the term “regulatory sequences” includes promoters, enhancers, and other elements that may control gene expression. Standard molecular biology textbooks such as Sambrook et al. eds “Molecular Cloning: A Laboratory Manual” 3rd ed., Cold Spring Harbor Press (2001) may be consulted to design suitable expression vectors that may further include an origin of replication and selectable gene markers. It should be recognized, however, that the choice of a suitable expression vector and the combination of functional elements therein depends upon multiple factors including the choice of the host cell to be transformed and/or the type of protein to be expressed.

The term “fragment” or “portion” as used herein can refer to, for example, an at least about 10, 20, 50, 75, 100, 150, 200, 250, 300, 500, 1000, 2000, 5000, 6,000, 8,000, 10,000, 20,000, 30,000, 40,000, 50,000 or 60,000 nucleotide long portion of a nucleic acid (e.g., cDNA) that has been formed artificially (e.g., by chemical synthesis) or, for example, formed by cleaving a natural product into multiple pieces, or by using restriction endonucleases or mechanical shearing, or formed enzymatically, for example, by PCR or any other polymerizing technique known in the art, or, for example, formed by a recombination event in a host cell or from recombinant nucleic acid technology known to one of skill in the art. The term “fragment” or “portion” as used herein may also refer to, for example, an at least about 5, 10, 20, 30, 40, 50, 75, 100, 150, 200, 250, 300, 400, 500, 1000, 2000, 5000, 6,000, 8,000 or 10,000 amino acid portion of an amino acid sequence, which portion is cleaved from a naturally occurring amino acid sequence by proteolytic cleavage by at least one protease, or is a portion of the naturally occurring amino acid sequence synthesized by chemical methods or using recombinant DNA technology (e.g., expressed from a portion of the nucleotide sequence encoding the naturally occurring amino acid sequence) known to one of skill in the art. “Fragment” or “portion” may also refer to a portion, for example, of about 5%, about 10%, about 20%, about 30%, about 40%, about 50%, about 60%, about 70%, about 80% about 90% about 95% or about 99% of a particular nucleotide or amino acid sequence. A truncated sequence may be referred to as a fragment or portion of the sequence.

“Functional portion” or “functional fragment” as used herein means a portion or fragment of a whole capable of performing, in whole or in part, a function of the whole. For example, a biologically functional portion of a molecule means a portion of the molecule that performs a biological function of the whole or intact molecule. For example, a functional portion of a gene expression controlling region is a fragment or portion of the specified gene expression controlling region that, in whole or in part, regulates or controls gene expression (e.g., facilitates either in whole or in part) in a biological system (e.g., a promoter). Functional portions may be of any useful size. For example, a functional fragment may range in size from about 20 bases in length to a length equal to the entire length of the specified sequence minus one nucleotide. In another example, a functional fragment may range in size from about 50 bases in length to a length equal to the entire length of the specified sequence minus one nucleotide. In another example, a functional fragment may range in size from about 50 bases in length to about 70 kb in length. In another example, a functional fragment may range in size from about 500 bases in length to about 70 kb in length. In another example, a functional fragment may range in size from about 1 kb in length to about 70 kb in length. In another example, a functional fragment may range in size from about 1 kb in length to about 20 kb in length. In another example, a functional fragment may range in size from about 1 kb in length to about 10 kb in length. Functional portions may include, for example, and without limitation, one or more of a matrix attachment region, a transcription enhancer, a hormone responsive element or a CRI repeat element.

The terms “heterologous” and “exogenous” in general refer to a biomolecule such as a nucleic acid (e.g., a nucleotide coding sequence) or a protein that is not normally found in a certain cell or tissue contained in or produced by an organism. For example, a protein that is heterologous or exogenous to an egg is a protein that is not normally found in the egg.

The term “gene expression controlling region” as used herein refers to nucleotide sequences that are associated with a nucleotide sequence and which regulate, in whole or in part, the expression of the nucleotide sequence, for example, regulate, in whole or in part, the transcription of a nucleotide sequence. Exemplary transcription regulatory sequences include enhancer elements, hormone response elements, steroid response elements, negative regulatory elements, and the like. The “transcription regulatory sequences” may be incorporated into a nucleic acid vector to enable regulated transcription in certain cells. The “transcription regulatory sequence” may precede the region of a nucleic acid sequence that is in the region 5′ of the end of a protein coding sequence that may be transcribed into mRNA. Transcriptional regulatory sequences may also be located within a protein coding region, in regions of a gene that are identified as “intron” regions, or may be in other regions of nucleic acid sequence. In addition, “control gene expression,” or “controlling gene expression”, refers to regulation, in whole or in part, of the expression of a nucleotide sequence, for example, regulation, in whole or in part, of the transcription of a nucleotide sequence.

The term “genome” refers to the genetic material present in one or more cells of an animal such as an avian.

The term “immunoglobulin amino acid sequence” as used herein refers to an amino acid sequence of an immunoglobulin. An “immunoglobulin amino acid sequence” may be, but is not limited to, an immunoglobulin (preferably an antibody) heavy or light chain and may include a variable region, a diversity region, a joining region and/or a constant region or any combination, variant or truncated form thereof. The term “immunoglobulin amino acid sequences” further includes single-chain antibodies comprised of, but not limited to, an immunoglobulin heavy chain variable region, an immunoglobulin light chain variable region and optionally a peptide linker.

The term “isolated nucleic acid” as used herein refers to a nucleic acid that has been substantially removed from other components of the cell containing the nucleic acid or from other components of chemical/synthetic reaction used to generate the nucleic acid. In specific embodiments, the nucleic acid is 50%, 60%, 70%, 80%, 90%, 95%, 99% or 100% pure. The techniques used to isolate and characterize the nucleic acids and proteins of the present invention are well known to those of skill in the art and standard molecular biology and biochemical manuals may be consulted to select suitable protocols without undue experimentation. See, for example, Sambrook et al, 2001, Molecular Cloning: A Laboratory Manual, 3rd ed., Cold Spring Harbor Press; the content of which is incorporated herein by reference in its entirety.

The term “LTR component” as used herein refers to a retroviral LTR or a portion of a retroviral LTR. The term “promoter component” as used herein refers to a constitutive promoter or a portion of a constitutive promoter.

The term “nucleic acid” as used herein refers to any natural and synthetic linear and sequential arrays of nucleotides and nucleosides, for example cDNA, genomic DNA, mRNA, tRNA, oligonucleotides, oligonucleosides and derivatives thereof. Representative examples of the nucleic acids of the present invention include bacterial plasmid vectors including expression, cloning, cosmid and transformation vectors such as, but not limited to, plasmid vectors, animal viral vectors such as, but not limited to, modified adenovirus, influenza virus, polio virus, pox virus, retrovirus, and the like, vectors derived from bacteriophage nucleic acid, e.g., plasmids and cosmids, artificial chromosomes, such as but not limited to, Yeast Artificial Chromosomes (YACs) and Bacterial Artificial Chromosomes (BACs), and synthetic oligonucleotides like chemically synthesized DNA or RNA. The term “nucleic acid” further includes modified or derivatised nucleotides and nucleosides such as, but not limited to, halogenated nucleotides such as, but not only, 5-bromouracil, and derivatised nucleotides such as biotin-labeled nucleotides.

The term “nucleic acid vector” or “vector” as used herein refers to a natural or synthetic single or double stranded plasmid or viral nucleic acid molecule, or any other nucleic acid molecule, such as, but not limited, to viral vectors (e.g., vectors derived from retroviruses), YACs, BACs, bacteriophage-derived artificial chromosome (BBPAC), cosmid or P1 derived artificial chromosome (PAC), that can be transfected or transformed into cells and replicate independently of, or within, the host cell genome. A circular double stranded vector can be linearized by treatment with an appropriate restriction enzyme based on the nucleotide sequence of the vector.

The terms “operably linked” or “operatively linked” refer to the configuration of the coding and control sequences so as to perform the desired function. Thus, control sequences operably linked to a coding sequence are capable of effecting the expression of the coding sequence and/or regulating in which tissues, at what developmental time points, or in response to which signals a coding sequence is expressed. For example, a coding sequence is operably linked to or under the control of transcriptional regulatory regions in a cell when DNA polymerase will bind the promoter sequence and transcribe the coding sequence into mRNA that can be translated into the encoded protein. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence. Such intervening sequences include but are not limited to enhancer sequences which are not transcribed or are not bound by polymerase.

The terms “percent sequence identity” or “percent sequence homology” or “percent sequence similarity” as used herein refer to the degree of sequence matching between two nucleic acid sequences or two amino acid sequences, for example, as determined using the algorithm of Karlin & Attschul (1990) Proc. Natl. Acad. Sci. 87: 2264-2268, modified as in Karlin & Attschul (1993) Proc. Natl. Acad. Sci. 90: 5873-5877. Such an algorithm is incorporated into the NBLAST and XBLAST programs of Attschul et al. (1990) T. Mol. Biol. Q15: 403-410. BLAST nucleotide searches are performed with the NBLAST program, score=100, wordlength=12, to obtain nucleotide sequences homologous to a nucleic acid molecule of the invention. BLAST protein searches are performed with the XBLAST program, score=50, wordlength=3, to obtain amino acid sequences homologous to a reference amino acid sequence. To obtain gapped alignments for comparison purposes, Gapped BLAST is utilized as described in Attschul et al. (1997) Nucl. Acids Res. 25: 3389-3402. When utilizing BLAST and Gapped BLAST programs, the default parameters of the respective programs (e.g. XBLAST and NBLAST) are used. Other algorithms, programs and default settings may also be suitable such as, but not only, the GCG-Sequence Analysis Package of the U.K. Human Genome Mapping Project Resource Centre that includes programs for nucleotide or amino acid sequence comparisons.

“Therapeutic proteins” or “pharmaceutical proteins” include an amino acid sequence which in whole or in part makes up a drug. A pharmaceutical composition can include one or more pharmaceutical proteins or therapeutic proteins.

The terms “polynucleotide” and “nucleic acid sequence” are used interchangeably herein and include, but are not limited to, coding sequences (polynucleotide(s) or nucleic acid sequence(s) which are transcribed and translated into amino acid sequence in vitro or in vivo when placed under the control of appropriate regulatory or control sequences); control sequences (e.g., translational start and stop codons, promoter sequences, ribosome binding sites, polyadenylation signals, transcription factor binding sites, transcription termination sequences, upstream and downstream regulatory domains, enhancers, silencers, and the like); and regulatory sequences (DNA sequences to which a transcription factor(s) binds and alters the activity of a gene's promoter either positively (induction) or negatively (repression)).

The term “probe” as used herein, when referring to a nucleic acid, refers to a nucleotide sequence that can be used to hybridize with and thereby identify the presence of a complementary sequence, or a complementary sequence differing from the probe sequence but not to a degree that prevents hybridization under the hybridization stringency conditions used. The probe may be modified with labels such as, but not only, radioactive groups, biotin, and the like that are well known in the art.

The terms “recombinant nucleic acid” and “recombinant DNA” as used herein refer to a combination of at least two nucleic acids that is not naturally found in a eukaryotic or prokaryotic cell in a particular configuration. The nucleic acids may include, but are not limited to, nucleic acid vectors, gene expression regulatory elements, origins of replication, suitable gene sequences that when expressed confer antibiotic resistance, protein-encoding sequences and the like. The term “recombinant amino acid sequence” is meant to include an amino acid sequence produced by recombinant DNA techniques such that it is distinct from a naturally occurring amino acid sequence either in its location, purity or structure. Generally, such a recombinant amino acid sequence will be present in a cell in an amount different from that normally observed in nature.

The term “sense strand” as used herein refers to a single stranded DNA molecule from a genomic DNA that may be transcribed into RNA which may be translated into an amino acid sequence product. The term “antisense strand” as used herein refers to the single strand DNA molecule of a genomic DNA that is complementary to the sense strand of the gene.

The terms “transformation” and “transfection” as used herein refer to the process of inserting a nucleic acid into a host. Many techniques are well known to those skilled in the art to facilitate transformation or transfection of a nucleic acid into a prokaryotic or eukaryotic organism. These methods involve a variety of techniques, such as treating the cells with high concentrations of salt such as, but not only, a calcium or magnesium salt, an electric field, detergent, or liposome mediated transfection, to render the host cell competent for the uptake of the nucleic acid molecules, and by such methods as sperm-mediated and restriction-mediated integration.

As used herein, a “transgenic animal” is any non-human animal, such as an avian species, including a chicken, in which one or more of the cells of the animal contain a heterologous nucleic acid introduced by way of human intervention, such as by transgenic techniques well known in the art. The nucleic acid is introduced into a cell, directly or indirectly by introduction into a precursor of the cell, by way of deliberate genetic manipulation, such as by microinjection or by infection with a recombinant virus. The term genetic manipulation typically refers to the introduction of a recombinant DNA molecule into an organism. This molecule may be integrated into a chromosome, or it may be extrachromosomally replicating DNA. In the typical transgenic animal, the transgene causes cells to express a recombinant form of the subject amino acid sequence, e.g. either agonistic or antagonistic forms, or in which the gene has been disrupted. In certain embodiments, the genome of the animal has been modified such that a heterologous gene expression element is inserted so as to be operably linked to an endogenous coding sequence. The terms “chimeric animal” or “mosaic animal” are used herein to refer to animals in which a recombinant nucleotide sequence is found, or in which the recombinant gene is expressed in some but not all cells of the animal. The term “tissue-specific” indicates that the recombinant nucleotide sequence is present and/or expressed in some tissues but not others.

As used herein, the term “transgene” means a nucleic acid sequence (encoding, for example, a amino acid sequence normally present in a human) that is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location that differs from that of the natural gene or its insertion results in a knockout). A trangene can also include one or more regulatory sequences designed to be inserted into the genome such that the transgene regulates the expression of a coding sequence, e.g., to increase expression and/or to change the timing and or tissue specificity of expression.

This description uses nomenclature accepted by the Cucurbit Genetics Cooperative as it appears in the Cucurbit Genetics Cooperative Report 18:85 (1995), incorporated herein by reference in its entirety.

### ABBREVIATIONS

Abbreviations used herein include the following: aa, amino acid(s); bp, base pair(s); ml, milliliter; min, minute(s); nt, nucleotide(s); SSC, sodium chloride-sodium citrate; ug, microgram(s); ul, microliter(s); uM, micromolar; UTR, untranslated region.

### DETAILED DESCRIPTION

The invention is directed to hybrid promoters and the production and use of the promoters. In one embodiment, the invention is directed to the use of hybrid promoters to produce exogenous proteins in avians, though the invention is not limited thereto. For example, the promoters may be useful in animals other than avians and may be useful in cell lines which are grown in culture.

In one embodiment, the invention is drawn to hybrid promoters wherein the hybrid promoters comprise a retroviral LTR (long terminal repeat) component linked to a promoter component. In a particularly useful embodiment of the invention, the 3′ end of the LTR component is linked to the 5′ end of the promoter component to form the hybrid promoter which is operably linked to a coding sequence. The term LTR component as used herein refers to a retroviral LTR or a portion of a retroviral LTR. The term promoter component as used herein refers to a constitutive promoter or a portion of a constitutive promoter. However, the invention contemplates hybrid promoters comprising an LTR component linked at its 3′ end to the 5′ end of any useful promoter or a portion of any useful promoter, for example, ovalbumin, ovomucoid, ovoinhibitor, conalbumin, ovomucin and lysozyme promoters or portions thereof.

In certain embodiments, the LTR component is a truncated LTR or an LTR with an internal portion of the LTR removed. In one useful embodiment, the LTR is truncated by removing a portion of the 3′ end of the LTR.

Particularly useful LTR components employed in hybrid promoters of the invention include LTR components obtained from retroviruses such as oncogenic viruses. Examples of such LTR components include, without limitation, LTR components obtained from Rous Sarcoma Virus (RSV), Murine Leukemia Virus (MLV), Molony Murine Sarcoma Virus (MMSV), Moloney Murine Leukemia Virus (MMLV), Avian Leukosis Virus (ALV) and lentivirus (e.g., human immunodeficiency virus (HIV), feline immunodeficiency virus (FIV), bovine immunodeficiency virus (BIV) and simian immunodeficiency virus (SIV)). Other useful LTRs are contemplated for use in the present invention such as LTRs from retrotransposons.

In certain embodiments, the promoter component is a truncated constitutive promoter or a constitutive promoter with an internal portion of the promoter removed. In one useful embodiment, the constitutive promoter is truncated by removing a portion of the 5′ end of the promoter.

Examples of promoter components which may be employed in hybrid promoters of the invention include, without limitation, promoter components obtained from the cytomegalovirus (CMV) promoter, ef-1a promoter, PGK promoter (phosphoglycerate kinase) and a beta actin promoter (e.g., chicken beta actin promoter, human beta actin promoter and bovine beta actin promoter). In one particular embodiment, the promoter components employed in the hybrid promoters of the invention are viral constitutive promoter components.

Typically, in hybrid promoters of the invention, the 3′ end of the LTR component is linked to the 5′ end of the promoter component, i.e., linked by a phosphodiester bond.

Reducing, changing or eliminating polyadenylation signal (AATAAA) of the LTR portion of hybrid promoters of the invention may produce a particularly useful promoter. It is believed a vector containing such a hybrid promoter would provide for an increased titer of vector recovered from the packaging cell line. For example, such an altered vector may decrease premature transcription termination that could occur in an otherwise similar or identical vector. Examples of possible changes that can be made to the polyA AATAAA sequence are changing the sequence to AACAAA or AATATT, for example, and simply truncating the LTR to remove the polyA sequence.

In hybrid promoters of the invention, typically the LTR component and the promoter component are linked as a linear DNA molecule in close proximity to each other. For example, though not exclusively, the LTR component and the promoter component are separated by less than about 100 nucleotides. For example, the LTR component and the promoter component can be separated by less than about 90 nucleotides, less than about 80 nucleotides, less than about 70 nucleotides, less than about 60 nucleotides, less than about 50 nucleotides, less than about 40 nucleotides, less than about 30 nucleotides, less than about 20 nucleotides, less than about 10 nucleotides or less than about 5 nucleotides. In certain useful embodiments, the LTR component and the promoter component are separated by no intervening nucleotides.

A nucleotide sequence from a vector containing the RSV LTR and CMV promoter, with an intervening nucleotide sequence between the two, is shown in FIG. 1A and FIG. 1B. The invention contemplates hybrid promoters containing all or a portion of the LTR in FIG. 1B and all or a portion of the promoter in FIG. 1B. In one embodiment, the hybrid promoter of the invention comprises the nucleotide sequence of FIG. 1B with the nucleotide sequence spanning from about nucleotide 347 to about nucleotide 2488 deleted. In another embodiment, the hybrid promoter of the invention comprises the nucleotide sequence of FIG. 1B with the nucleotide sequence spanning from about nucleotide 360 to about nucleotide 2460 deleted. In another embodiment, the hybrid promoter of the invention comprises the nucleotide sequence of FIG. 1B with the nucleotide sequence spanning from about nucleotide 239 to about nucleotide 2820 deleted. In another embodiment, the hybrid promoter of the invention comprises the nucleotide sequence of FIG. 1B with the nucleotide sequence spanning from about nucleotide 347 to about nucleotide 2950 deleted. In another embodiment, the hybrid promoter of the invention comprises the nucleotide sequence of FIG. 1B with the nucleotide sequence spanning from about nucleotide 245 to about nucleotide 2950 deleted. In one particularly useful embodiment, the hybrid promoter of the invention comprises the nucleotide sequence of FIG. 1B with the nucleotide sequence spanning from about nucleotide 262 to about nucleotide 2822 deleted. In one particular embodiment, the hybrid promoter is a fusion of the 3′ end of the U5 and R regions of the RSV LTR linked to the 5′ end of a CMV promoter. In one embodiment, all or a portion of the enhancer region of the CMV promoter is removed in a hybrid promoter of the invention. In one embodiment, the transcription start site of the RSV LTR component and the CMV promoter component are both present such that the hybrid promoter has two potential transcription start sites.

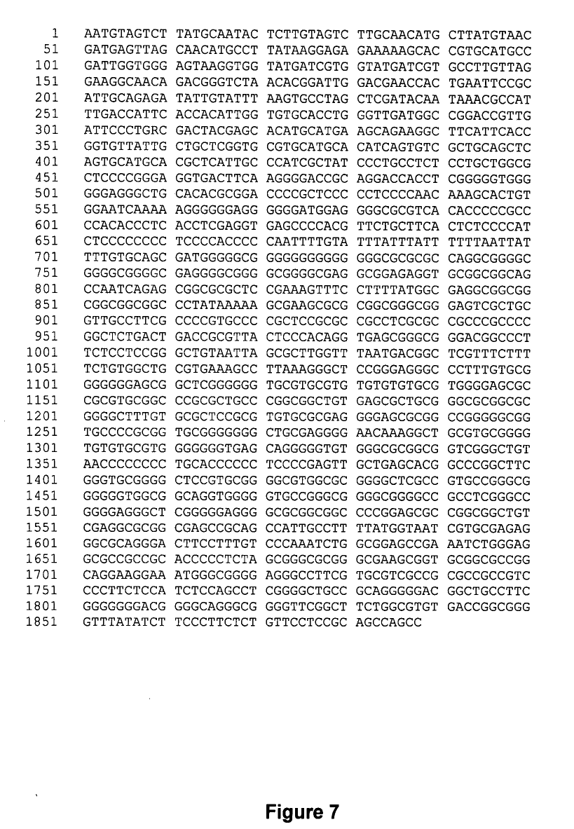

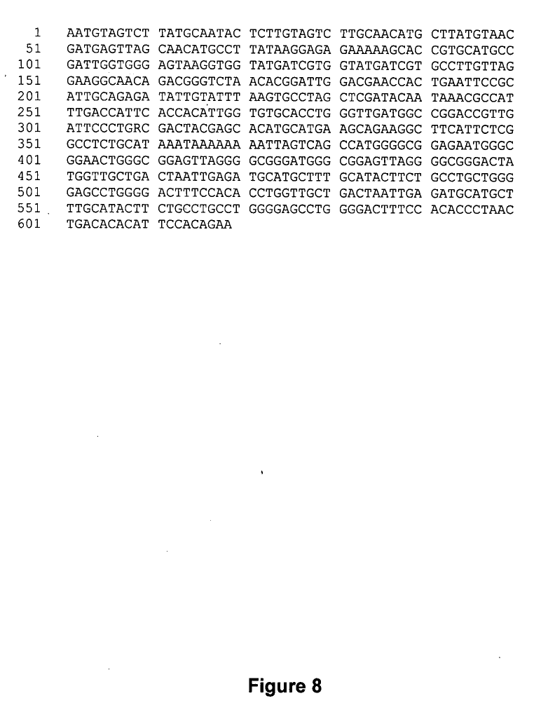

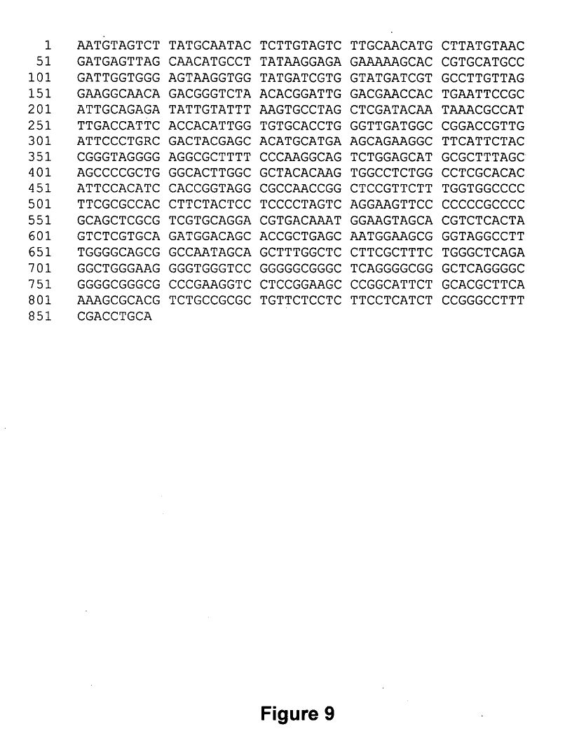

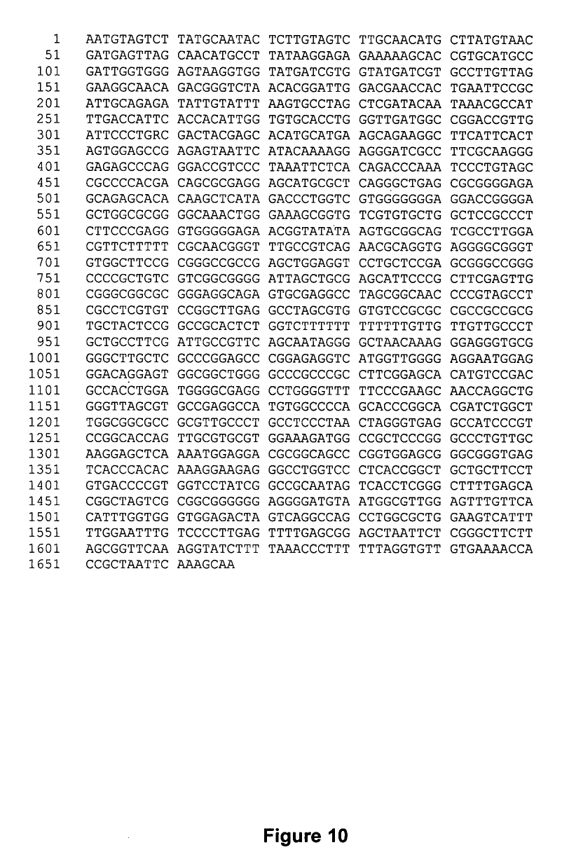

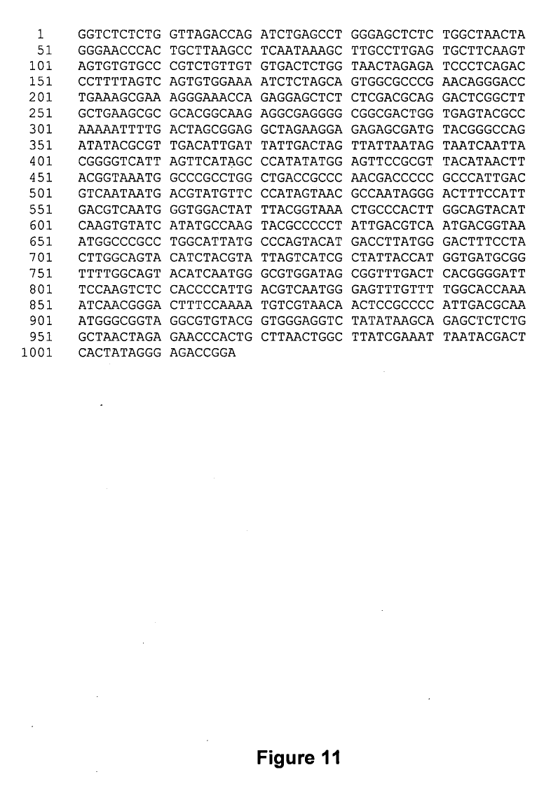

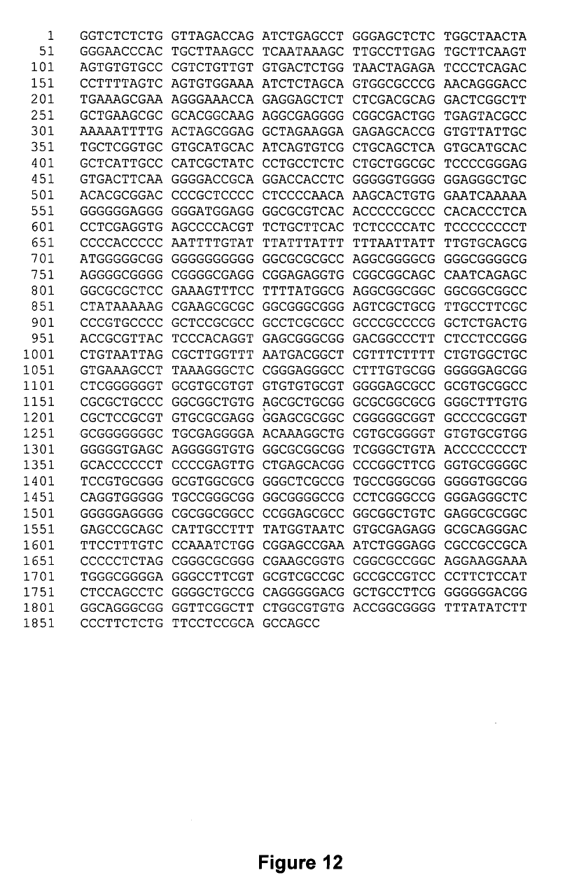

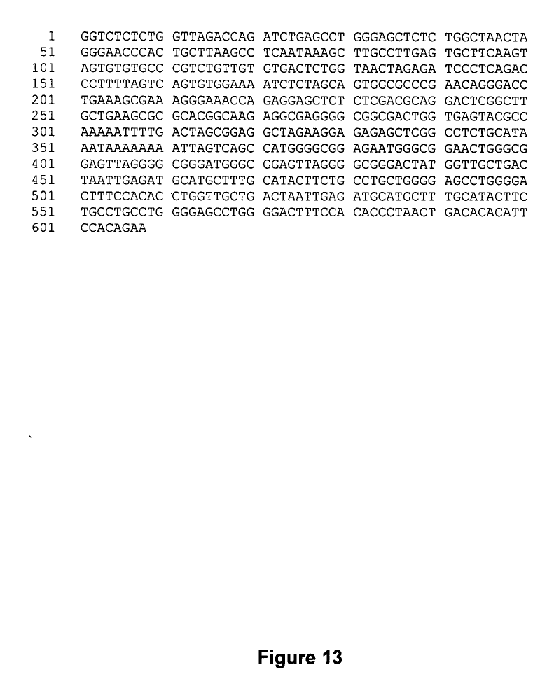

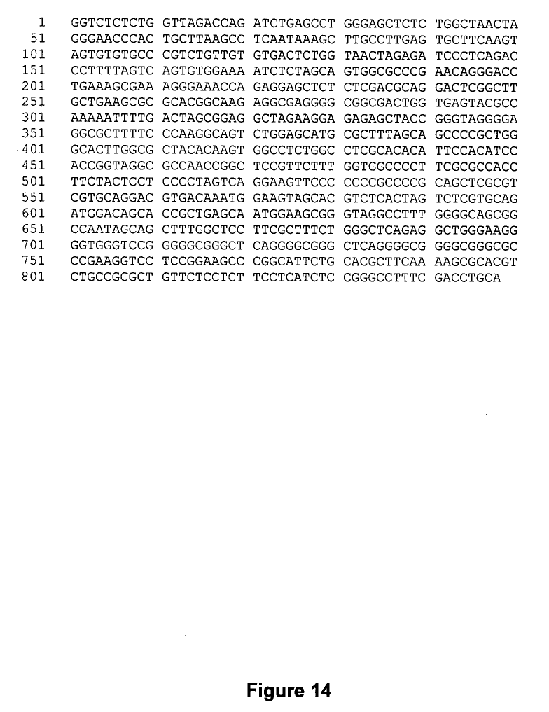

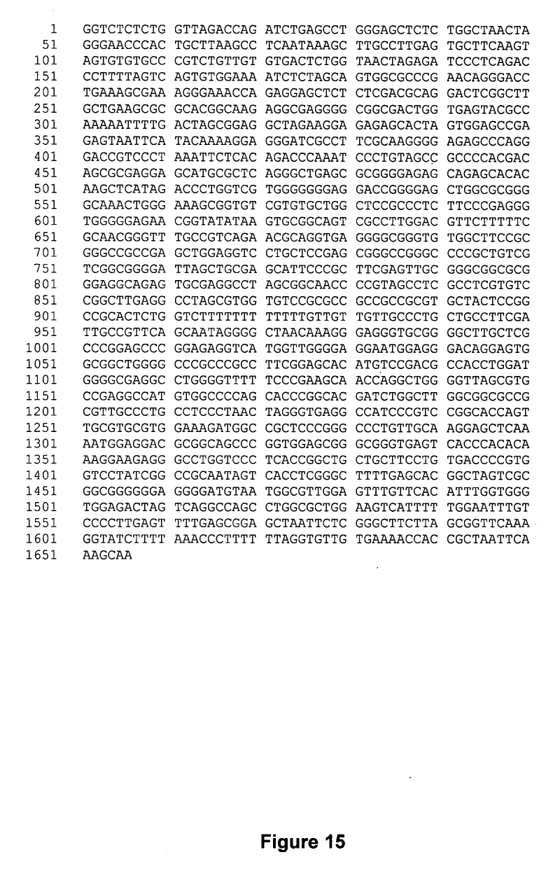

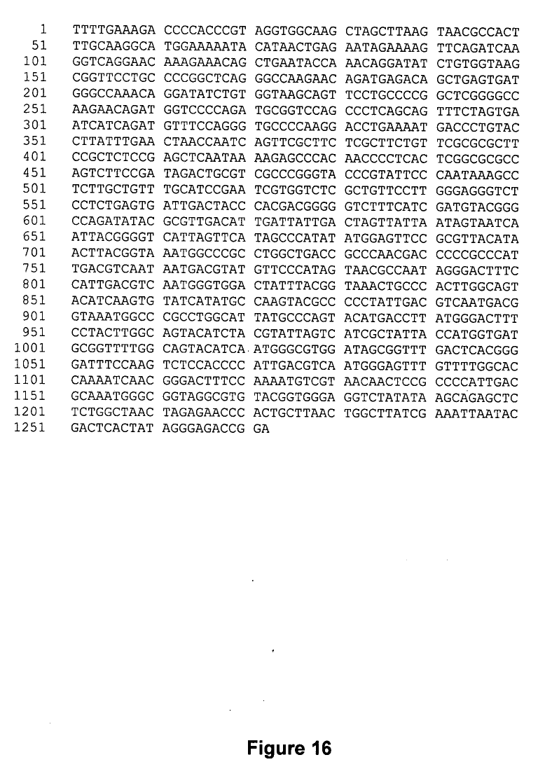

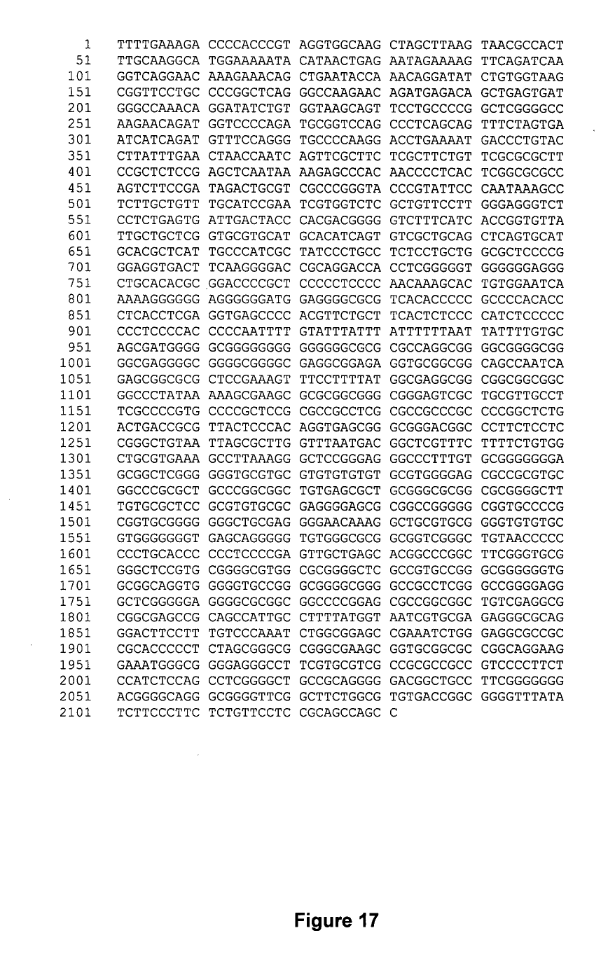

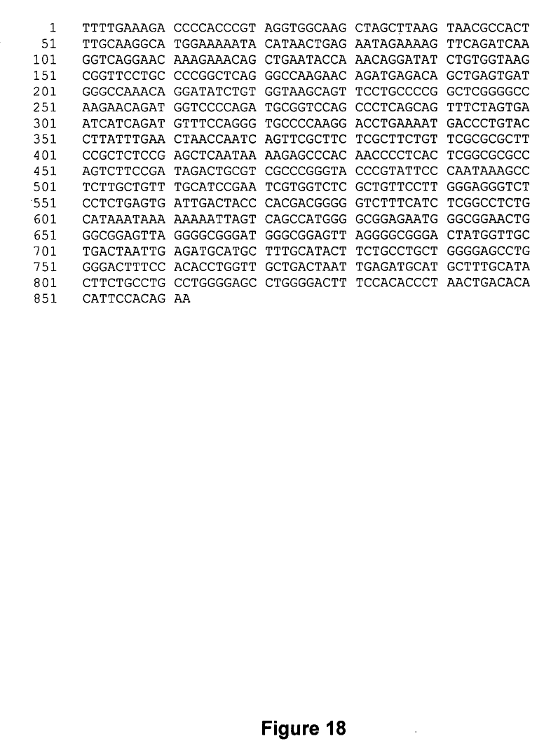

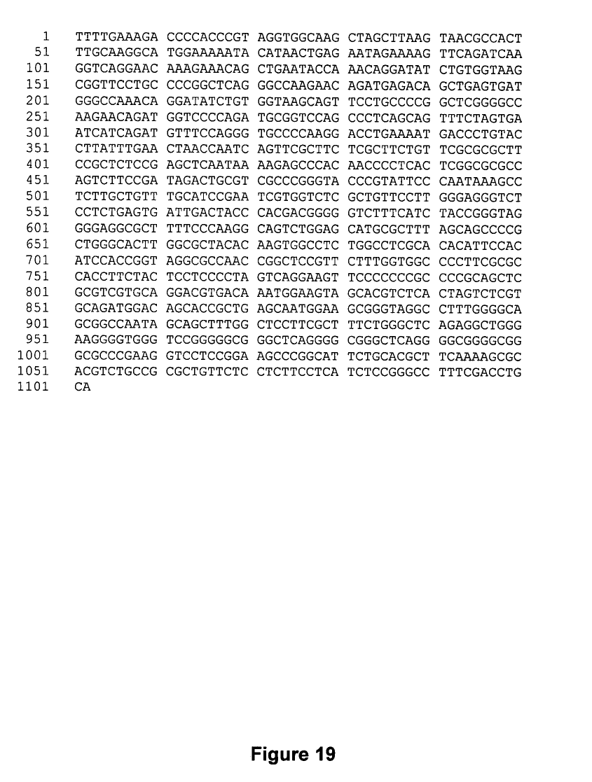

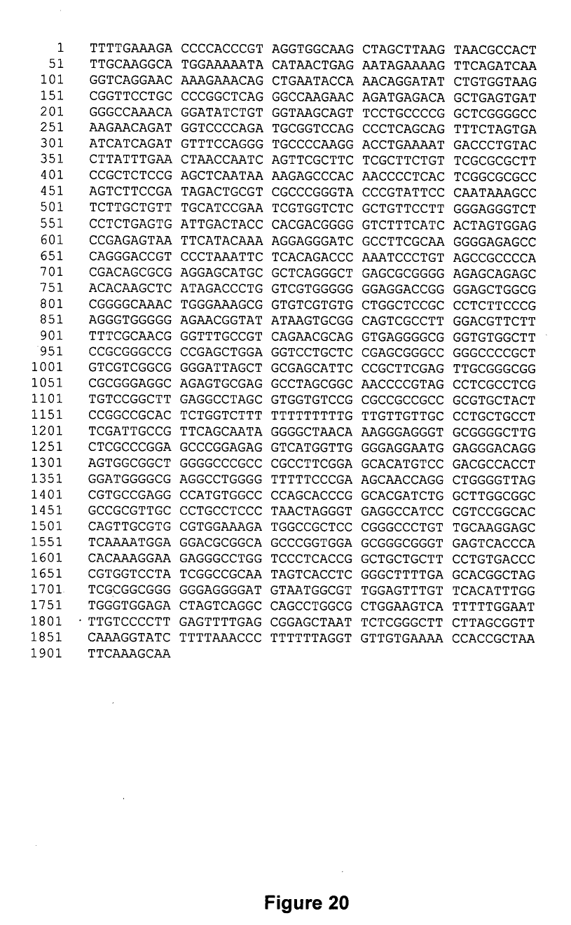

Examples of certain hybrid promoters are shown in FIGS. 7 to 20. Hybrid promoters of the invention include the RSV LTR linked to beta actin promoters such as the chicken beta actin promoter (FIG. 7, junction at nucleotides 346-347), the RSV LTR linked to the SV 40 promoter (FIG. 8, junction at nucleotides 346-347), the RSV LTR linked to the PGK promoter (FIG. 9, junction at nucleotides 346-347), the RSV LTR linked to the mouse EF1 alpha promoter (FIG. 10, junction at nucleotides 346-347), HIV (human immunodeficiency virus) LTR linked to a CMV promoter (FIG. 11, junction at nucleotides 335-336), the HIV LTR linked to a chicken beta actin promoter (FIG. 12, junction at nucleotides 335-336), the HIV LTR linked to the SV 40 promoter (FIG. 13, junction at nucleotides 335-336), the HIV LTR linked to a PGK promoter (FIG. 14, junction at nucleotides 335-336), the HIV LTR linked to the mouse EF1 alpha promoter (FIG. 15, junction at nucleotides 335-336), the MMLV LTR linked to a CMV promoter (FIG. 16, junction at nucleotides 589-590), the MMLV LTR linked to a chicken beta actin promoter (FIG. 17, junction at nucleotides 589-590), the MMLV LTR linked to the SV 40 promoter (FIG. 18, junction at nucleotides 589-590), the MMLV LTR linked to a PGK promoter (FIG. 19, junction at nucleotides 589-590) and the MMLV LTR linked to a mouse EF1 alpha promoter (FIG. 20, junction at nucleotides 589-590). The invention contemplates truncation of each of the LTRs and promoters used in hybrid promoters of the invention including those shown in FIGS. 7 to 20. For example, each of the hybrid promoters may be truncated by about 20 nucleotides on each side of the junction truncating both the promoter and LTR within the hybrid promoter. In another example, each of the hybrid promoters may be truncated by about 50 nucleotides on each side of the junction. In still another example, each of the hybrid promoters may be truncated by about 100 nucleotides on each side of the junction. In still another example, each of the hybrid promoters may be truncated by about 200 nucleotides or more on each side of the junction.

In other embodiments only the promoter component of the hybrid promoter is truncated. For example, the promoter component contained in the hybrid promoters of the invention shown in FIGS. 7 to 20 may be truncated by about 20 nucleotides 3′ of the junction. In another example, the promoter component contained in these hybrid promoters may be truncated by about 50 nucleotides 3′ of the junction. In still another example, the promoter component contained in these hybrid promoters may be truncated by about 100 nucleotides 3′ of the junction. In still another example, the promoter component contained in these hybrid promoters may be truncated by about 200 nucleotides or more 3′ of the junction.

In still other embodiments only the LTR component is truncated. For example, the LTR component contained in the hybrid promoters shown in FIGS. 7 to 20 may be truncated by about 20 nucleotides 5′ of the junction. In another example, the LTR component contained in these hybrid promoters may be truncated by about 50 nucleotides 5′ of the junction. In still another example, the LTR component contained in these hybrid promoters may be truncated by about 100 nucleotides 5′ of the junction. In still another example, the LTR component contained in these hybrid promoters may be truncated by about 200 nucleotides 5′ of the junction.

One useful hybrid promoter (i.e., capable of high protein production activity in transgenic chickens) of the invention was analyzed by TFSEARCH revealing a number of potential transcription factor binding sites. The hybrid promoter (termed herein 407-Promoter) can be delineated in FIG. 1B wherein the 3′ end of the RSV LTR component corresponding to nucleotides 1 to 261 in FIG. 1B is linked to the 5′ end of the CMV promoter component corresponding to nucleotides 2823 to 3169.

Transcription Factor Binding Sites in the LTR region of the 407-Promoter were identified through TFSEARCH: Searching Transcription Factor Binding Sites (ver 1.3) and are listed below. Sites marked with an asterisk are also present in the CMV component of the hybrid promoter.
chicken vitellogenin promoter-binding protein*
CCAAT/enhancer binding factor*
C/EBPalpha*
C/EBPbeta*
Serum responsive factor
AP-1*
GATA-binding factor 1*
delta-crystallin enhancer-binding protein deltaEF1
Cdx-1*
cellular and viral CCAAT box*
nuclear factor Y (Y-box binding factor)
alcohol dehydrogenase gene regulator*

Additional transcription Factor Binding Sites in the CMV promoter component of the 407-Promoter that are not found in the LTR component of the 407-Promoter identified through TFSEARCH: Searching Transcription Factor Binding Sites (ver 1.3) are listed below.
c-Rel
NF-kappaB
octamer-binding factor 1

The close proximity of the transcription factor binding sites of the LTR component and promoter component in the 407-Promoter may yield an increased rate of transcription initiation compared to if the transcription start sites are not in relative close proximity. Therefore, without wishing to limit the invention to any particular theory or mechanism of operation, it is believed that in hybrid promoters of the invention, one or more transcription factor binding sites of the LTR component may work in cooperation with one or more transcription factor binding sites of the 3′ promoter component of the hybrid promoter when the LTR component and the 3′ promoter component are covalently linked within close proximity to one another providing for particularly strong promoters.

Hybrid promoters of the invention encompass nucleotide sequences that are 80%, 85%, 90%, 95%, 96%, 97%, 98% and 99% identical to the nucleotide sequence of each hybrid promoter disclosed herein. It is assumed the promoters disclosed herein are double stranded DNA molecules even though only one strand of the DNA may be discussed or shown.

In one particularly useful embodiment, the hybrid promoters of the invention are contemplated for the production of useful RNAs and proteins. For example, the promoters can be operably linked to coding sequences which are transcribed or transcribed and translated in vivo or in vitro. In one embodiment, promoters of the invention are used for the production of proteins which in certain cases may be foreign (i.e., exogenous or heterologous) to the cell or animals in which the proteins are produced.

The hybrid promoters disclosed herein are contemplated for application in any useful cell type. For example, the promoters can be used in cells grown in culture such as CHO cells, human cell lines such as HeLa cells and avian cell lines such as chicken fibroblast cells lines (e.g., DF-1 cells). Methods of producing cell lines containing heterologous DNA are well known in the art and are within the scope of the invention. In one embodiment, proteins may be produced in transgenic animals such as sheep, cow, goat and pig. In one particularly useful embodiment, promoters of the invention are employed in avians (e.g., chicken, quail and duck).

Methods for production of transgenic animals are well known in the art and can be used for introducing a transgene containing a hybrid promoter of the invention into an animal cell. Aspects of the production of transgenic avians are disclosed in U.S. patent application Ser. No. 11/708,598, filed Feb. 20, 2007 and U.S. patent application Ser. No. 11/542,093, filed Oct. 3, 2006, the disclosures of which are incorporated in their entirety herein by reference. However, other methods are available and known in the art to introduce a desired DNA sequence into animals such as avians (e.g., chickens) and as such the invention is not limited to producing transgenic animals or avians according to any particular method or methods.

Eukaryotic cells, such as vertebrate cells, which are useful for the production of heterologous protein that contain a promoter of the invention operably linked to a desired coding sequence are within the scope of the invention. In a particularly useful embodiment of the invention, the hybrid promoters of the invention are employed in tubular gland cells of an avian. Therefore, the invention includes oviduct cells (e.g., tubular gland cells) of egg laying species such as avians (e.g., chicken, quail, turkey) which contain hybrid promoters of the invention. The oviduct cells may be grown in vitro or may be present in a live avian.

Any useful method for introducing the hybrid promoters of the invention into a cell (e.g., cell in culture or cell of a transgenic animal) is contemplated for use herein. For example, the promoters may be contained in a vector in the cell or the promoters may be integrated into the genome of the cell in which they employed. Any useful vector for use in conjunction with promoters of the invention is within the scope of the invention. For example, the invention includes plasmid vectors, artificial chromosome vectors, and viral vectors including, without limitation, viral vectors derived from Rous Sarcoma Virus (RSV), Murine Leukemia Virus (MLV), Molony Murine Sarcoma Virus (MMSV), Moloney Murine Leukemia Virus (MMLV), Avian Leokosis Virus (ALV) and lentivirus (e.g., human immunodeficiency virus (HIV)), feline immunodeficiency virus (FIV), bovine immunodeficiency virus (BIV) and simian immunodeficiency virus (SIV)). The use of certain vectors such as retroviral vectors will typically, although not exclusively, result in integration of the desired transgene, which includes the hybrid promoter, into the host cell genome.

The invention contemplates all useful vectors for introducing promoters of the invention into cells including avian embryo cells. For example, the invention contemplates truncation of the 5′ LTR of the retroviral vector used to introduce hybrid promoters of the invention into cells to reduce transcription from the LTR which may interfere with the transcription of the hybrid promoter, a phenomenon termed promoter interference. Therefore, in one embodiment, the LTR promoter is contained on a self-inactivating vector (SIN vector) as is understood in the art. Production of certain self-inactivating vectors is disclosed, for example, in Flamant et al, J Gen Virol, January 1993; 74 (Pt 1):39-46 and Ilves et al, Gene, Jun. 1, 1996; 171(2):203-8. The disclosure of each of these two references is incorporated herein in its entirety by reference. For example, the 3′LTR of the vector (distinguished from the LTR component) can be truncated. During integration, the truncated 3′LTR sequence is copied to the 5′LTR, inactivating the LTR thereby reducing or eliminating transcription from the 5′LTR.

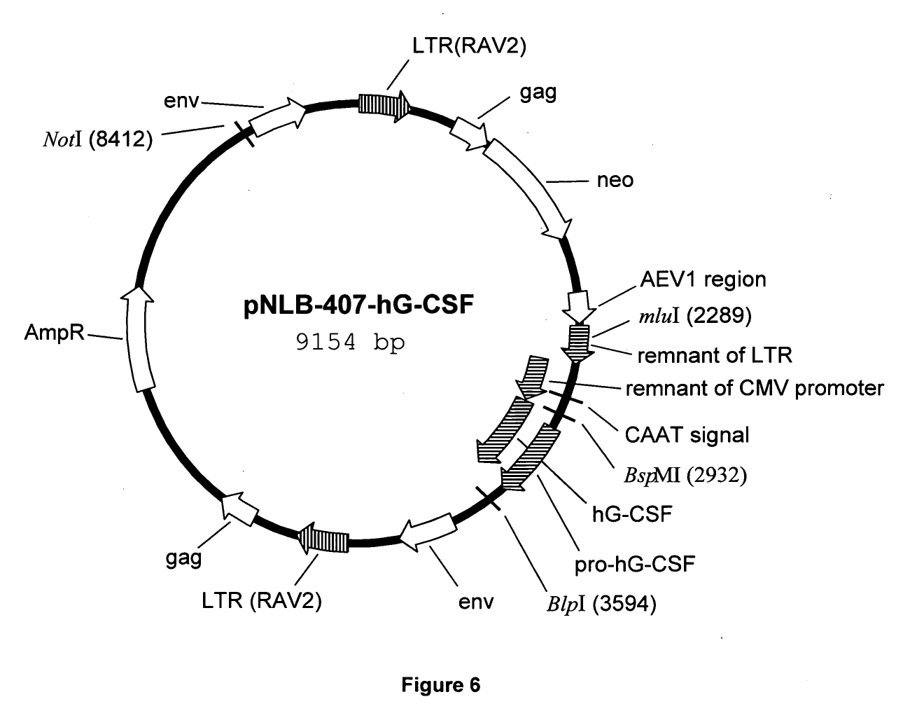

The vector containing the 407-Promoter described in Example 2 used in the production of G-CSF is a particularly useful self-inactivating (SIN) vector. It may be that in certain circumstances the truncated LTR in the vector may still be capable of disrupting the activity of the downstream promoter, though to a lesser degree than the LTR of a non-SIN vector. In addition, the presence of three LTR sequences, as seen in FIG. 6, may in certain instances reduce the titer and stability of the retroviral vector. A new vector termed the Tombak vector, which can remedy these deficiencies, has been designed and produced as described in Example 4.

The 407-Promoter consists of the intact ALV U3 region and about 16 basepairs of the 5′ ALV R region fused to a portion of the CMV promoter. Thus about 5 bp of the R region is missing and the U5 region is replaced with a portion of the CMV promoter sequence. The invention contemplates the use of a retroviral vector in which a promoter component is substituted for a portion of the 5′ LTR, such as the U5 portion of the LTR. Additional sequence of the LTR may also be replaced by the promoter component. In such a vector the LTR having the deletion is functional in two aspects: 1) during integration of the provirus into the host genome; 2) as an LTR component to form a functional hybrid promoter. An example of such a vector is the Tombak vector.

The hybrid promoters of the invention provide for a facilitated production of heterologous or exogenous protein in cell culture or in transgenic animals relative to the production of protein in a similar or identical cell culture or transgenic animal in which the complete promoter that corresponds to the promoter component of the hybrid promoter is employed. For example, hybrid promoters of the invention provide for an increased production of exogenous or heterologous protein. In one embodiment, the invention is drawn and a transgenic chicken (e.g., a G1 transgenic chicken), and to eggs laid by the chicken, that can lay an egg containing greater than 20 micrograms of protein encoded by a transgene per ml of egg white. For example, the invention is drawn to a transgenic chicken which can lay an egg containing between about 20 micrograms of protein encoded by a transgene per ml of egg white and about 20 milligrams of protein encoded by a transgene per ml of egg white. In another example, the invention is drawn to a transgenic chicken which can lay an egg containing between about 20 micrograms of protein encoded by a transgene per ml of egg white and about 6 milligrams of protein encoded by a transgene per ml of egg white. In another example, the invention is drawn to a transgenic chicken which can lay an egg containing between about 20 micrograms of protein encoded by a transgene per ml of egg white and about 3 milligrams of protein encoded by a transgene per ml of egg white. In another example, the invention is drawn to a transgenic chicken which can lay an egg containing between about 20 micrograms of protein encoded by a transgene per ml of egg white and about 2 milligrams of protein encoded by a transgene per ml of egg white. In another example, the invention is drawn to a transgenic chicken which can lay an egg containing between about 20 micrograms of protein encoded by a transgene per ml of egg white and about 1 milligram of protein encoded by a transgene per ml of egg white. In another example, the invention is drawn to a transgenic chicken which can lay an egg containing between about 10 micrograms of protein encoded by a transgene per ml of egg white and about 5 milligrams of protein encoded by a transgene per ml of egg white. In another example, the invention is drawn to a transgenic chicken which can lay an egg containing between about 10 micrograms of protein encoded by a transgene per ml of egg white and about 3 milligrams of protein encoded by a transgene per ml of egg white. In another example, the invention is drawn to a transgenic chicken which can lay an egg containing between about 50 micrograms of protein encoded by a transgene per ml of egg white and about 4 milligrams of protein encoded by a transgene per ml of egg white. In another example, the invention is drawn to a transgenic chicken which can lay an egg containing between about 40 micrograms of protein encoded by a transgene per ml of egg white and about 4 milligrams of protein encoded by a transgene per ml of egg white. In another example, the invention is drawn to a transgenic chicken which can lay an egg containing between about 20 micrograms of protein encoded by a transgene per ml of egg white and about 0.5 milligrams of protein encoded by a transgene per ml of egg white. It is understood that typically a chicken egg contains approximately 30 ml of egg white per egg. Therefore, since approximate quantities of exogenous protein per ml of egg white are specifically disclosed, approximate quantities of exogenous protein per chicken egg are also specifically disclosed. For example, an egg containing between about 20 micrograms of protein encoded by a transgene per ml of egg white and about 0.5 milligrams of protein encoded by a transgene per ml of egg white would contain between about 0.6 milligrams and about 15 milligrams of exogenous protein. The invention also includes methods of preparing or isolating the exogenous proteins from eggs produced in accordance with the invention, as is understood in the art of protein purification.

The invention can be used to express, in large yields and at low cost, desired proteins including those used as human and animal pharmaceuticals, diagnostics, and livestock feed additives. For example, the invention includes transgenic avians that produce such proteins and eggs laid by the transgenic avians which contain the protein, for example, in the egg white. The present invention is contemplated for use in the production of any desired protein including pharmaceutical or therapeutic proteins.

The production of human proteins as disclosed herein is of particular interest. The human form of each of the proteins disclosed herein for which there is a human form, is contemplated for production in accordance with the invention.

Proteins contemplated for production as disclosed herein include, but are not limited to, fusion proteins, growth hormones, cytokines, structural proteins and enzymes including human growth hormone, interferon, lysozyme, and β-casein, albumin, α-1 antitrypsin, antithrombin III, collagen, factors VIII, IX, X (and the like), fibrinogen, insulin, lactoferrin, protein C, erythropoietin (EPO), granulocyte colony-stimulating factor (G-CSF), granulocyte macrophage colony-stimulating factor (GM-CSF), tissue-type plasminogen activator (tPA), somatotropin, and chymotrypsin. Modified immunoglobulins and antibodies, including immunotoxins which bind to surface antigens on human tumor cells and destroy them, can also be produced as disclosed herein.

Other specific examples of therapeutic proteins which may be produced as disclosed herein include, without limitation, factor VIII, b-domain deleted factor VIII, factor VIIa, factor IX, anticoagulants, hirudin, alteplase, tpa, reteplase, tpa, tpa-3 of 5 domains deleted, insulin, insulin lispro, insulin aspart, insulin glargine, long-acting insulin analogs, hgh, glucagons, tsh, follitropin-beta, fsh, gm-csf, pdgh, ifn alpha2, ifn alpha2a, ifn alpha2b, inf-apha, inf-beta 1b, ifn-beta 1a, ifn-gamma1b, il-2, il-11, hbsag, ospa, murine mab directed against t-lymphocyte antigen, murine mab directed against tag-72, tumor-associated glycoprotein, fab fragments derived from chimeric mab directed against platelet surface receptor gpII(b)/III(a), murine mab fragment directed against tumor-associated antigen ca125, murine mab fragment directed against human carcinoembryonic antigen, cea, murine mab fragment directed against human cardiac myosin, murine mab fragment directed against tumor surface antigen psma, murine mab fragments (fab/fab2 mix) directed against hmw-maa, murine mab fragment (fab) directed against carcinoma-associated antigen, mab fragments (fab) directed against nca 90, a surface granulocyte nonspecific cross reacting antigen, chimeric mab directed against cd20 antigen found on surface of b lymphocytes, humanized mab directed against the alpha chain of the il2 receptor, chimeric mab directed against the alpha chain of the il2 receptor, chimeric mab directed against tnf-alpha, humanized mab directed against an epitope on the surface of respiratory synctial virus, humanized mab directed against her 2, human epidermal growth factor receptor 2, human mab directed against cytokeratin tumor-associated antigen anti-ctla4, chimeric mab directed against cd 20 surface antigen of b lymphocytes domase-alpha dnase, beta glucocerebrosidase, tnf-alpha, il-2-diptheria toxin fusion protein, tnfr-lgg fragment fusion protein laronidase, dnaases, alefacept, darbepoetin alfa (colony stimulating factor), tositumomab, murine mab, alemtuzumab, rasburicase, agalsidase beta, teriparatide, parathyroid hormone derivatives, adalimumab (lgg1), anakinra, biological modifier, nesiritide, human b-type natriuretic peptide (hbnp), colony stimulating factors, pegvisomant, human growth hormone receptor antagonist, recombinant activated protein c, omalizumab, immunoglobulin e (lge) blocker, lbritumomab tiuxetan, ACTH, glucagon, somatostatin, somatotropin, thymosin, parathyroid hormone, pigmentary hormones, somatomedin, erythropoietin, luteinizing hormone, chorionic gonadotropin, hypothalmic releasing factors, etanercept, antidiuretic hormones, prolactin and thyroid stimulating hormone.

The invention includes methods for producing multimeric proteins including immunoglobulins, such as antibodies, and antigen binding fragments thereof. Thus, in one embodiment of the present invention, the multimeric protein is an immunoglobulin, wherein the first and second heterologous polypeptides are immunoglobulin heavy and light chains respectively.

In certain embodiments, an immunoglobulin polypeptide encoded by the transcriptional unit of at least one expression vector may be an immunoglobulin heavy chain polypeptide comprising a variable region or a variant thereof, and may further comprise a D region, a J region, a C region, or a combination thereof. An immunoglobulin polypeptide encoded by an expression vector may also be an immunoglobulin light chain polypeptide comprising a variable region or a variant thereof, and may further comprise a J region and a C region. The present invention also contemplates multiple immunoglobulin regions that are derived from the same animal species, or a mixture of species including, but not only, human, mouse, rat, rabbit and chicken. In certain embodiments, the antibodies are human or humanized.

In other embodiments, the immunoglobulin polypeptide encoded by at least one expression vector comprises an immunoglobulin heavy chain variable region, an immunoglobulin light chain variable region, and a linker peptide thereby forming a single-chain antibody capable of selectively binding an antigen.

Examples of therapeutic antibodies that may be produced in methods of the invention include, but are not limited, to HERCEPTIN™ (Trastuzumab) (Genentech, CA) which is a humanized anti-HER2 monoclonal antibody for the treatment of patients with metastatic breast cancer; REOPRO™ (abciximab) (Centocor) which is an anti-glycoprotein IIb/IIIa receptor on the platelets for the prevention of clot formation; ZENAPAX™ (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath; Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); ICM3 is a humanized anti-ICAM3 antibody (ICOS Pharm); IDEC-114 is a primate anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (CS) antibody (Alexion Pharm); D2E7 is a humanized anti-TNF-α antibody (CATIBASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); CAT-152, a human anti-TGF-β₂ antibody (Cambridge Ab Tech); Cetuximab (BMS) is a monoclonal anti-EGF receptor (EGFr) antibody; Bevacizuma (Genentech) is an anti-VEGF human monoclonal antibody; Infliximab (Centocore, JJ) is a chimeric (mouse and human) monoclonal antibody used to treat autoimmune disorders; Gemtuzumab ozogamicin (Wyeth) is a monoclonal antibody used for chemotherapy; and Ranibizumab (Genentech) is a chimeric (mouse and human) monoclonal antibody used to treat macular degeneration.

In certain instances it may be desirable to add a sialic acid or other sugar molecule to a therapeutic protein produced by the transgenic avians (e.g., transgenic chickens), for example, to extend the biological half life of the therapeutic protein. For example, it may be advantageous to sialate a therapeutic protein such as a cytokine, e.g., erythropoietin, produced in a transgeneic chicken. The sialic acid may be linked to the protein by an enzymatic reaction as is understood in the art or by chemical addition as understood in the art. In one embodiment, the sialic acid is added to one or more glycosylation structures present on the therapeutic protein produced by a transgenic chicken. Addition of sialic acid to the protein may increase efficacy of the protein for therapeutic use.

While it is possible that, for use in therapy, therapeutic proteins produced in accordance with this invention may be administered in raw form, it is preferable to administer the therapeutic proteins as part of a pharmaceutical formulation.

The invention thus further provides pharmaceutical formulations comprising poultry derived glycosylated therapeutic proteins or a pharmaceutically acceptable derivative thereof together with one or more pharmaceutically acceptable carriers thereof and, optionally, other therapeutic and/or prophylactic ingredients and methods of administering such pharmaceutical formulations. The carrier(s) must be “acceptable” in the sense of being compatible with the other ingredients of the formulation and not deleterious to the recipient thereof. Methods of treating a patient (e.g., quantity of pharmaceutical protein administered, frequency of administration and duration of treatment period) using pharmaceutical compositions of the invention can be determined using standard methodologies known to physicians of skill in the art.

Pharmaceutical formulations include those suitable for oral, rectal, nasal, topical (including buccal and sub-lingual), vaginal or parenteral. The pharmaceutical formulations include those suitable for administration by injection including intramuscular, sub-cutaneous and intravenous administration. The pharmaceutical formulations also include those for administration by inhalation or insufflation. The formulations may, where appropriate, be conveniently presented in discrete dosage units and may be prepared by any of the methods well known in the art of pharmacy. The methods of producing the pharmaceutical formulations typically include the step of bringing the therapeutic proteins into association with liquid carriers or finely divided solid carriers or both and then, if necessary, shaping the product into the desired formulation.

Pharmaceutical formulations suitable for oral administration may conveniently be presented as discrete units such as capsules, cachets or tablets each containing a predetermined amount of the active ingredient; as a powder; as granules; as a solution; as a suspension; or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils) or preservatives.

Therapeutic proteins of the invention may also be formulated for parenteral administration (e.g., by injection, for example bolus injection or continuous infusion) and may be presented in unit dose form in ampoules, pre-filled syringes, small volume infusion or in multi-dose containers with an added preservative. The therapeutic proteins may be injected by, for example, subcutaneous injections, intramuscular injections, and intravenous infusions or injections.

The therapeutic proteins may take such forms as suspensions, solutions, or emulsions in oily or aqueous vehicles, and may contain formulatory agents such as suspending, stabilizing and/or dispersing agents. It is also contemplated that the therapeutic proteins may be in powder form, obtained by aseptic isolation of sterile solid or by lyophilization from solution, for constitution with a suitable vehicle, e.g., sterile, pyrogen-free water, before use.

For topical administration to the epidermis, the therapeutic proteins produced according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents or coloring agents.

Formulations suitable for topical administration in the mouth include lozenges comprising active ingredient in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical formulations suitable for rectal administration wherein the carrier is a solid are most preferably represented as unit dose suppositories. Suitable carriers include cocoa butter and other materials commonly used in the art, and the suppositories may be conveniently formed by a mixture of the active compound with the softened or melted carrier(s) followed by chilling and shaping in molds.

Formulations suitable for vaginal administration may be presented as pessaries, tampons, creams, gels, pastes, foams or sprays containing in addition to the active ingredient, such carriers as are known in the art to be appropriate.

For intra-nasal administration the therapeutic proteins of the invention may be used as a liquid spray or dispersible powder or in the form of drops.

Drops may be formulated with an aqueous or non-aqueous base also comprising one or more dispersing agents, solubilizing agents or suspending agents. Liquid sprays are conveniently delivered from pressurized packs.

For administration by inhalation, therapeutic proteins according to the invention may be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

For administration by inhalation or insufflation, the therapeutic proteins according to the invention may take the form of a dry powder composition, for example a powder mix of the compound and a suitable powder base such as lactose or starch. The powder composition may be presented in unit dosage form in, for example, capsules or cartridges or, e.g., gelatin or blister packs from which the powder may be administered with the aid of an inhalator or insufflator.

When desired, the above described formulations adapted to give sustained release of the active ingredient, may be employed.

The pharmaceutical compositions according to the invention may also contain other active ingredients such as antimicrobial agents, or preservatives.

In addition, it is contemplated that the therapeutic proteins of the invention may be used in combination with other therapeutic agents.

Compositions or compounds of the invention can be used to treat a variety of conditions. For example, there are many conditions for which treatment therapies are known to practitioners of skill in the art in which therapeutic proteins obtained from cell culture (e.g., CHO cells) are employed. The present invention contemplates that the therapeutic proteins produced in an avian system can be employed to treat such conditions. That is, the invention contemplates the treatment of conditions known to be treatable by conventionally produced therapeutic proteins by using therapeutic proteins produced in accordance with the invention. For example, erythropoietin produced in accordance with the invention can be used to treat human conditions such as anemia and kidney disease (e.g., chronic renal failure) and G-CSF produced in accordance with the invention can be used to treat cancer patients, each as is understood in the art.

Generally, the dosage administered will vary depending upon known factors such as age, health and weight of the recipient, type of concurrent treatment, frequency of treatment, and the like. Usually, a dosage of active ingredient can be between about 0.0001 milligrams and about 10 milligrams per kilogram of body weight. Precise dosage, frequency of administration and time span of treatment can be determined by a physician skilled in the art of therapeutic protein administration.

The present invention is further illustrated by the following examples, which are provided by way of illustration and should not be construed as limiting the scope of the invention. The contents of all references, published patents and patents cited throughout the present application are hereby incorporated by reference in their entireties.

### EXAMPLE 1

### Production of EPO Using a Hybrid Promoter

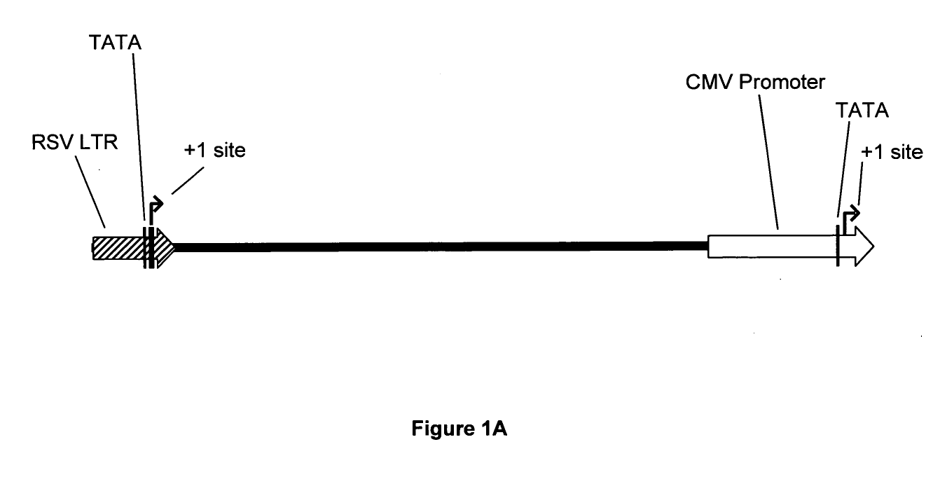

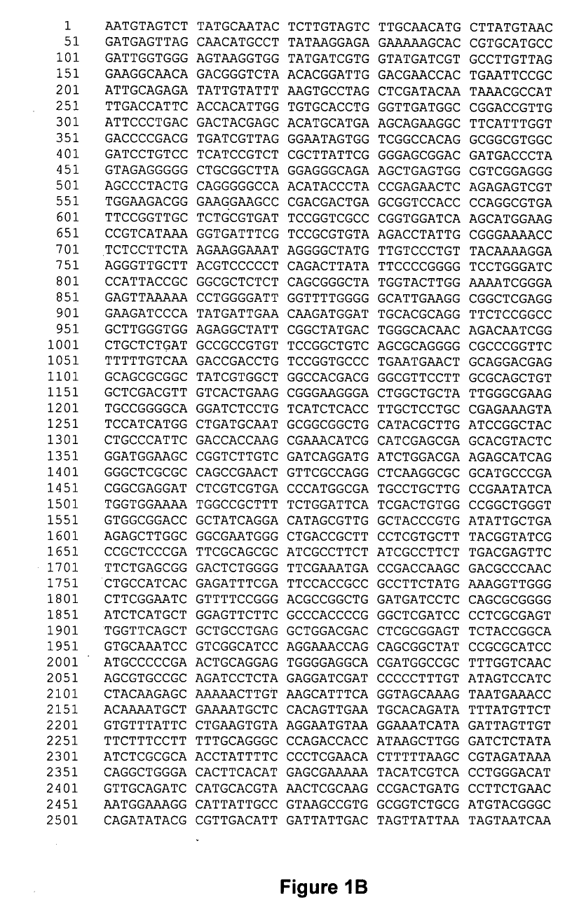

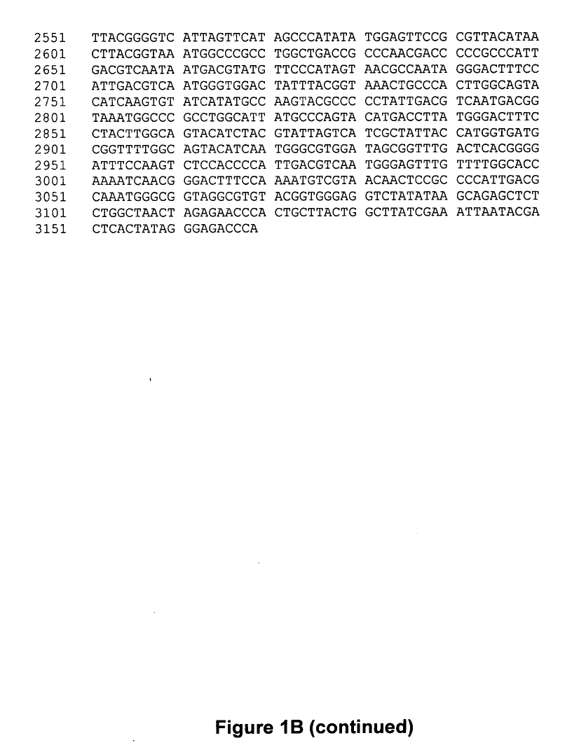

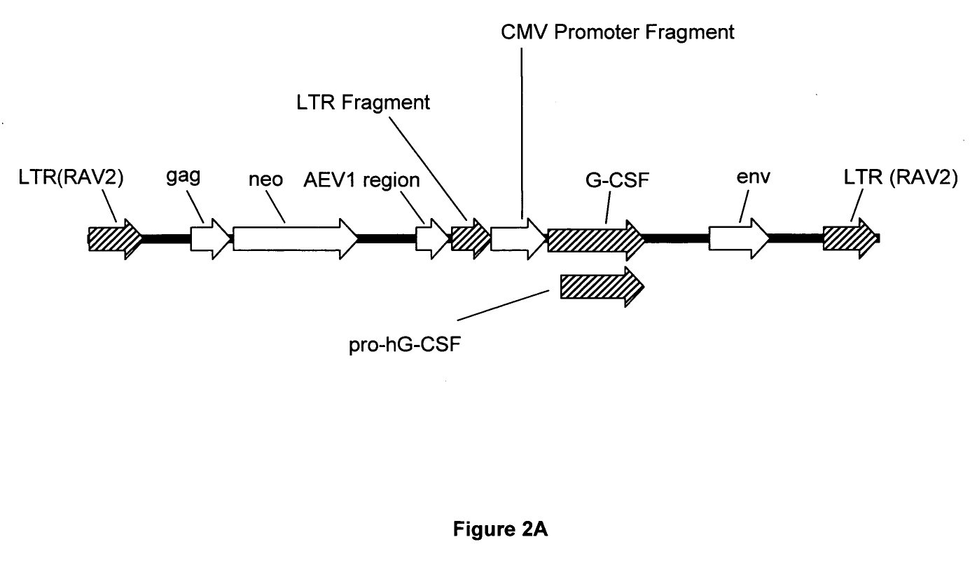

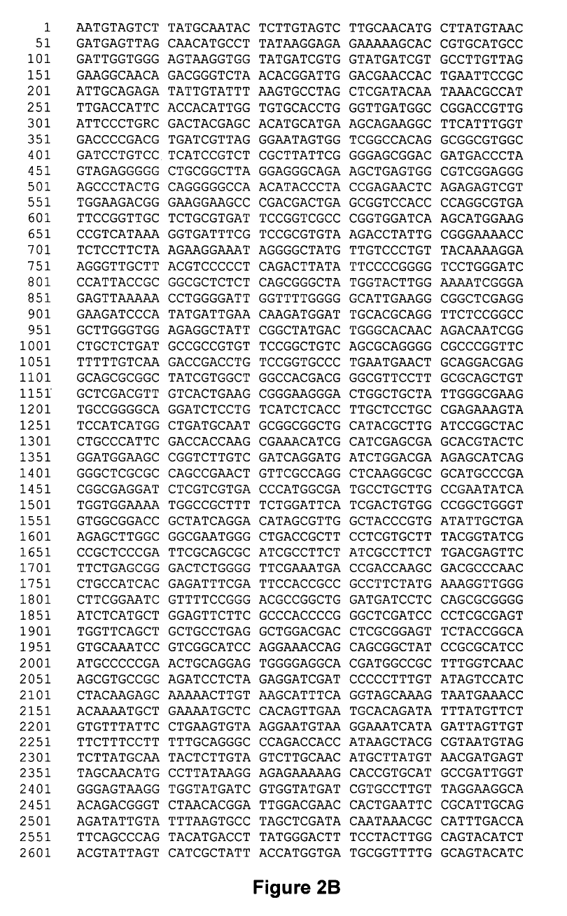

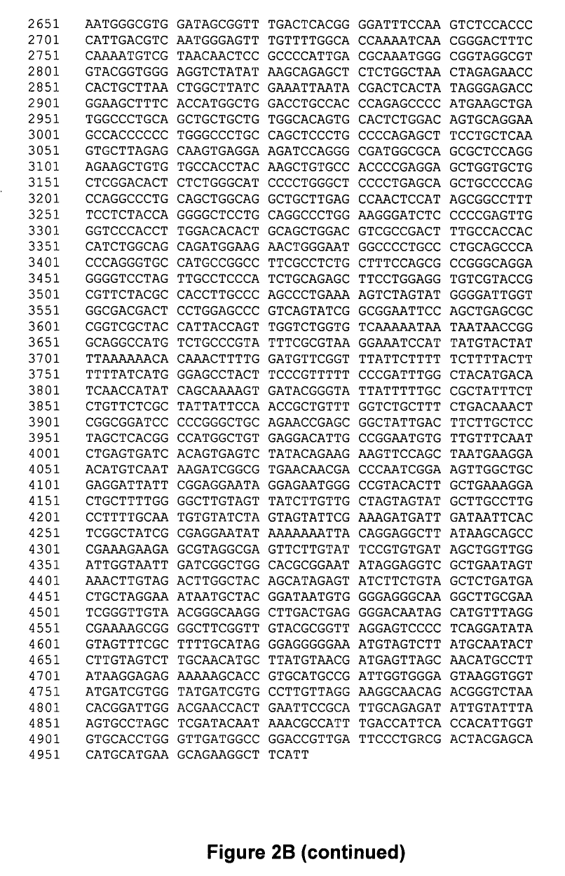

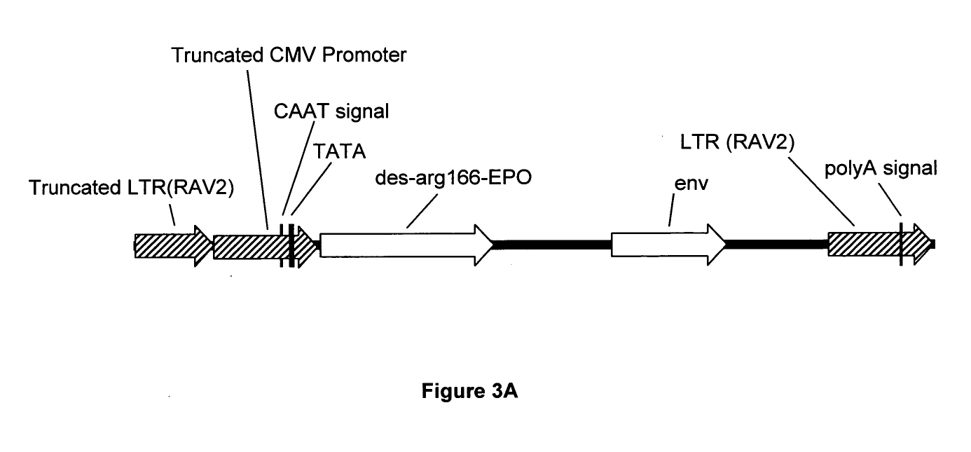

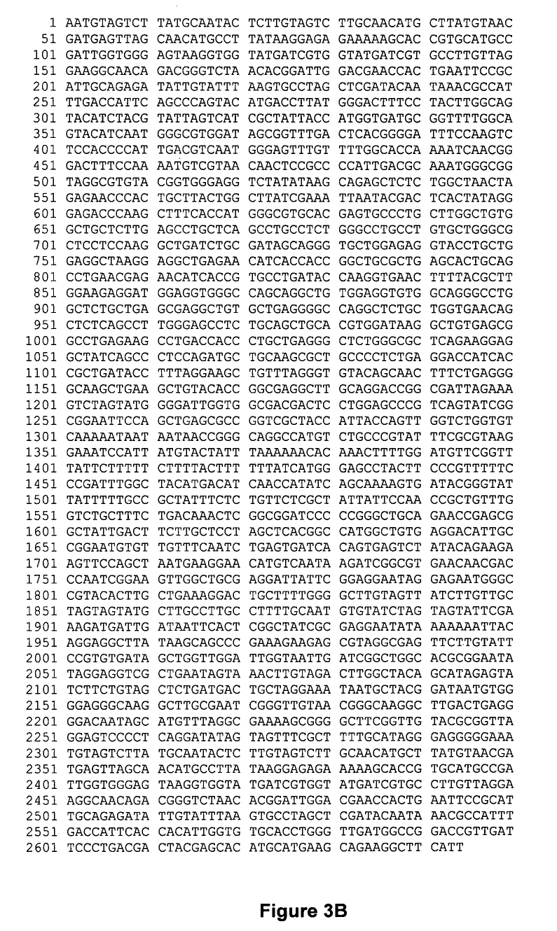

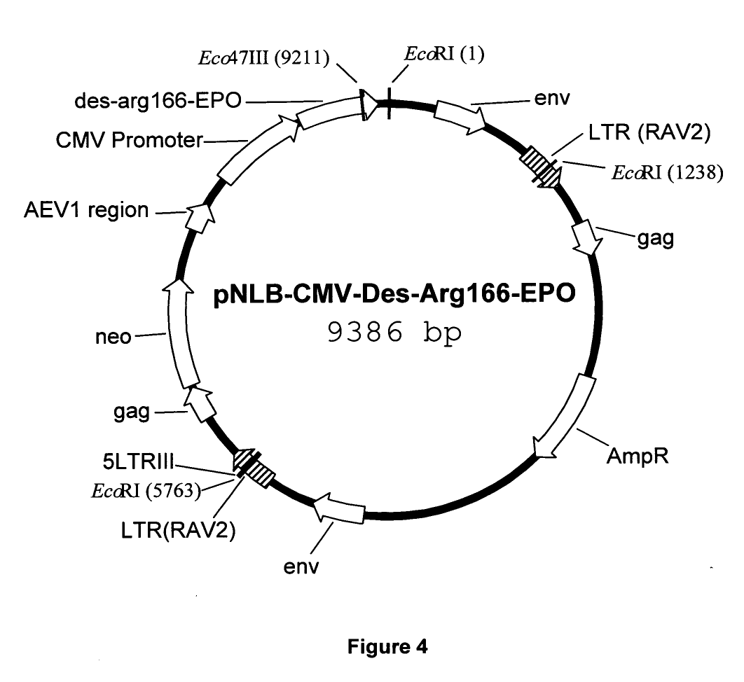

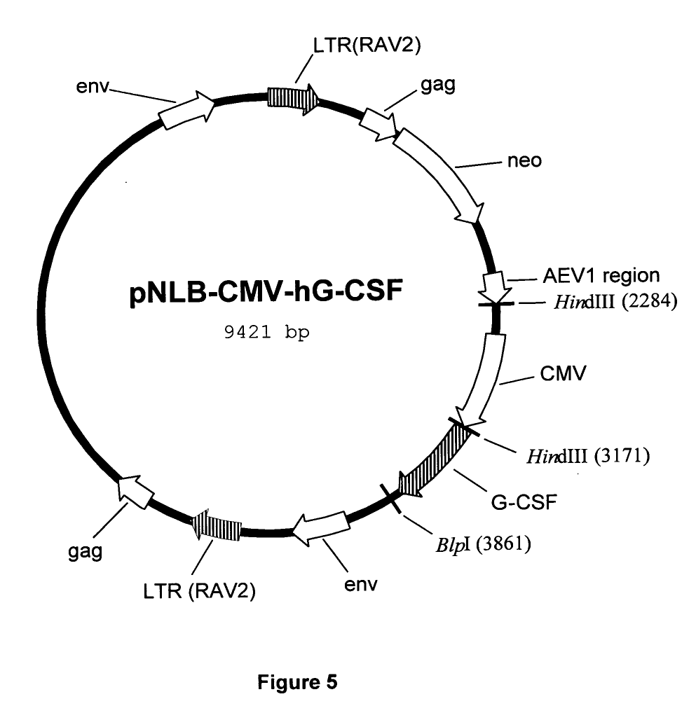

The transduction vector shown in FIG. 4, pNLB-CMV-Des-Arg166-EPO, was constructed as disclosed in U.S. patent application Ser. No. 11/454,399, filed Oct. 3, 2006, the disclosure of which is incorporated in its entirety herein by reference.

Virus particles were prepared from a chicken fibroblast cell line as disclosed in U.S. patent application Ser. No. 11/454,399. The virus particles were injected into the sub-germinal cavity of unincubated SPF White Leghorn embryos as follows. 7 ul of the virus suspension prepared according to Example 2 of U.S. patent application Ser. No. 11/454,399 was injected into the subgerminal cavity of 97 fertile, unincubated White Leghorn eggs (Charles River, SPAFAS). 54 chicks hatched and were reared to sexual maturity. Semen was collected and DNA extracted by the Chelex method. 100 ng of sperm DNA, as determined by the PicoGreen assay (Molecular Probes) was assayed for the presence of the EPO transgene using the Applied Biosystems TaqMan® Fast Universal PCR Master Mix and the Applied Biosystems 7900HT. The primers were:

(SEQ ID NO: 20)

SJ-EPO-for, 5′- GCCCTCCAGATGCTGCAA -3′
and

(SEQ ID NO: 21)

SJ-EPO-rev, 5′- CCCTAAACAGCTTCCTAAAGGTATCA -3′.

The Taqman EPO probe sequence was 5′-CGCTGCCCCTCTGAGGACCATC-3′ (SEQ ID NO: 22) and was labeled with FAM (6-carboxyfluorescin) at the 5′ end and TAMRA (N,N,N′,N′-tetramethyl-6-carboxyrhodamine) at the 3′end. One rooster was found to have a significant level of the EPO transgene in his semen. This rooster was bred to wildtype hens. Approximately 144 chicks were hatched. Their blood DNA was extracted and tested for the presence of the transgene using the EPO Taqman assay. Two chicks were found to be positive for the transgene. The level of the transgene was such that the cells of each of the G1 birds had one copy of the EPO transgene.

Expression levels of EPO of the G1 chicken designated 407 was 15 ug/ml in the blood and 100 ug/ml in the egg white, each as determined by ELISA.

The transgene present in hen 407 was analyzed by Southern blot analysis and was sequenced revealing the integrated transgene and nucleotide sequence shown in FIGS. 3A and 3B respectively which contains a hybrid promoter having a truncated LTR and a truncated CMV promoter. This hybrid promoter may be referred to herein the 407-Promoter. The 407-Promoter is the 3′ end of the RSV LTR fragment corresponding to nucleotides 1 to 261 in FIG. 1B linked to the 5′ end of the CMV promoter fragment corresponding to nucleotides 2823 to 3169 in FIG. 1B.

### EXAMPLE 2

### pSIN-407-hG-CSF Vector Construction

### Construction of pNLB-407-hG-CSF Vector from pNLB-CMV-hG-CSF

The CMV (cytomegalovirus) regulatory region of pNLB-CMV-hG-CSF, disclosed in Example 17 of U.S. patent application Ser. No. 11/708,598, filed Feb. 20, 2007, the disclosure of which is incorporated in its entirety herein by reference, was replaced by inserting a 620 bp Esp3I/HindIII PCR fragment containing the 407-Promoter into the HindIII/HindIII sites of pNLB-CMV-hG-CSF. First, an 887 bp fragment containing the CMV regulatory region of pNLB-CMV-hG-CSF was removed by HindIII digestion to yield a 8534 bp fragment. A 646 bp PCR fragment was obtained by amplifying the genomic DNA from the G1 chicken designated 407 of Example 1 using Hercules II Fusion DNA Polymerase (Stratagene, La Jolla, Calif.), and primers 407-pro-for (GGCGTCTCAAGCTACGCGTAATGTAGTCTTATGCAATACTCTTGTAGTC) (SEQ ID NO: 23) and 407-pro-rev (CGCCCATGGTGAAAGCTTCCGGTCTCCCTATA) (SEQ ID NO 24). The PCR was carried out in 25 ul with 100 ng genomic DNA, 250 uM each dNTP, 1× Hercules II Reaction Buffer, 0.5 ul Hercules II Fusion DNA Polymerase, 0.3 uM 407-pro-for, and 0.3 uM 407-pro-rev. The PCR steps were 95 C for 2 min; followed by 35 cycles of 95 C for 30 sec, 60 C for 30 sec, 72 C for 1 min; and the final extension at 72 C for 6 min. The PCR product was digested by Esp3I and HindIII to yield a 620 bp fragment. The 8534 bp fragment of pNLB-CMV-hG-CSF was treated with Calf Intestinal Alkaline Phosphatase (New England Biolabs, Beverly, Mass.), and ligated to the 620 bp PCR fragment to make pNLB-407-hG-CSF. The ligated product was transformed into ElectroMAX Stbl4 Cells (Invitrogen, Carlsbad, Calif.), and the transformants were screened by AatII digestion. The positive clone was analyzed by EcoRI digestion and sequenced.

The provirus region of pNLB-407-hG-CSF contains the hybrid promoter of Example 1 (407-Promoter) operably linked to the coding sequence of a protein of interest, in this case G-CSF, and the region of the circular vector spanning LTR to LTR is illustrated in FIGS. 2A and 2B. The G-CSF coding sequence can be substituted for other useful coding sequences, such as therapeutic protein coding sequences disclosed herein and other useful protein coding sequences as known to a practitioner of skill in the art.

### Construction of pSIN-407-hG-CSF Vector from pNLB-407-hG-CSF

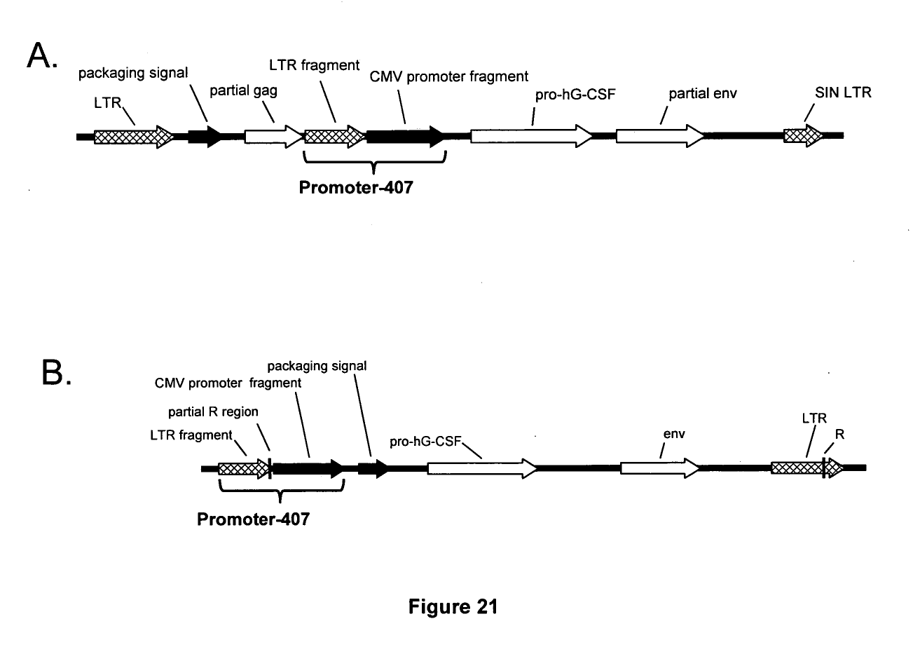

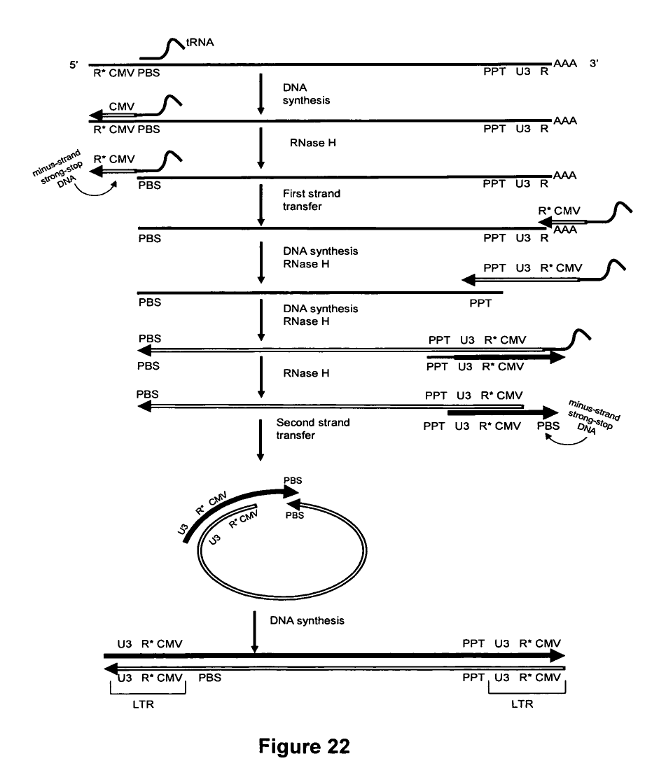

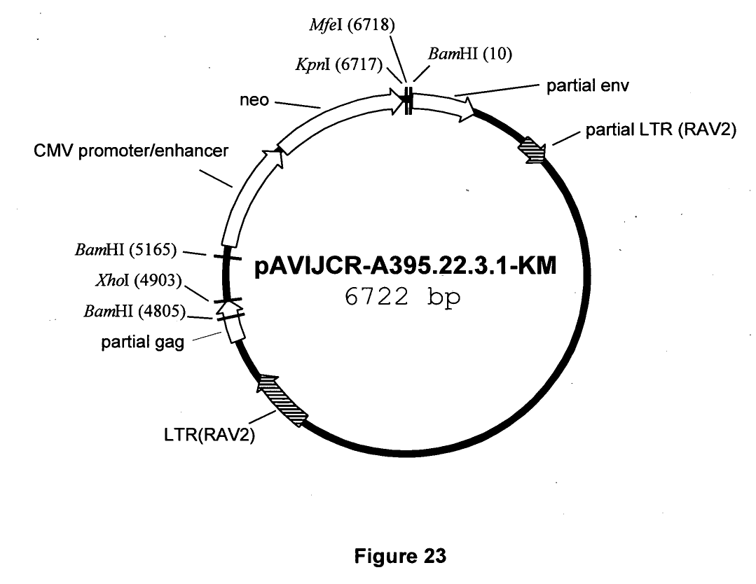

pAVIJCR-A395.22.3.1-KM shown in FIG. 23 and SEQ ID NO: 28 was cut with Mfe I and Xho I, filled in with Klenow and the 4911 bp fragment gel purified. pNLB-407-hG-CSF was cut with Mlu I and Blp I, filled in and the 1308 bp fragment ligated to the 4911 bp fragment from pAVIJCR-A395.22.3.1-KM to produce pSIN-407-hG-CSF. The portion of the circular pSIN-407-hG-CSF vector spanning LTR to LTR is shown in FIG. 21A. SEQ ID NO: 18 shows the complete nucleotide sequence of the circular pSIN-407-hG-CSF.

### Example 3

### Production of Translenic Chickens Expressing Human Granulocyte Colony Stimulating Factor (hG-CSF)

Production of transduction particles of pSIN-407-hG-CSF described in Example 2 was performed as described in Example 1 and the vector was introduced into chicken blastodermal cells in essentially the same manner as described for pNLB-CMV-Des-Arg166-EPO in Example 1. The embryos of 116 stage X eggs were injected with about 7 ul of pSIN-407-hG-CSF transduction particles without titering. 44 chicks hatched and were raised to sexual maturity. Five chicks tested positive for G-CSF, one of which was male.

DNA was extracted from sperm samples of each of the roosters by Chelex-100 extraction (Walsh et al., 1991). DNA samples were then subjected to Taqman™ analysis on a 7700 Sequence Detector (Perkin Elmer) using the primers SJ-G-CSF for (cagagcttcctgctcaagtgctta) (SEQ ID NO: 25) and SJ-G-CSF rev (ttgtaggtggcacacagcttct) (SEQ ID NO: 26) and the probe, SJ-G-CSF probe (agcaagtgaggaagatccagggcg) (SEQ ID NO: 27), to detect the transgene. The rooster with the highest level of the transgene in his sperm samples was bred to nontransgenic SPAFAS (White Leghorn) hens by artificial insemination. Blood DNA samples of the offspring were screened for the presence of the transgene by Taqman™ analysis as described above. Out of 2241 offspring, 47 G1s were found to be transgenic with 37 of 39 chicks serum positive for G-CSF (8 chicks untested). The positive chicks contained approximately 0.6 ug/ml to 12.8 ug/ml G-CSF in the serum, as measured by ELISA. Based on this it is expected that G-CSF in the egg white may be present between approximately 10 ug/ml and 120 ug/ml.

### EXAMPLE 4

### Production of the pTombak having a 5′ LTR Fragment which Functions in Retrovirus Integration and Serves as a Functional LTR Component of the Hybrid Promoter

FIG. 21B shows pTombak-hG-CSF having the 407-Promoter plus sequences required for retroviral packaging and integration. In this vector the packaging signal is inserted between the 407-Promoter and the coding sequence for the protein of interest. To create pTombak-hG-CSF, the 407 promoter, packaging signal region and a portion of the G-CSF coding sequence were synthesized and cloned into a standard plasmid vector. The vector was cut with Not I and BspMI and a 931 bp sequence containing the 407 promoter, packaging signal region and portion of the G-CSF coding sequence was isolated. pNLB-407-G-CSF was cut with BspMI and Not I, the 5480 bp fragment isolated and ligated to the 931 bp fragment, creating pTombak-hG-CSF. In the Tombak vector, the R region from about bp 17 to bp 21 (the R region is approximately 21 bp in length) and the U5 region of the 5′ LTR are replaced with the partial CMV promoter to produce the hybrid promoter which also serves as a functional LTR of the of the Tombak vector. The portion of the circular pTombak-hG-CSF vector spanning LTR to LTR is shown in FIG. 21B. The complete sequence for the pTombak-hG-CSF vector is shown in SEQ ID NO: 19.

### EXAMPLE 5

### Hybrid Promoter Activity Using pTombak

Chicken fibroblast cells were transduced with p407-G-CSF-SIN retroviral particles or the pTombak-hG-CSF retroviral particles, produced as disclosed in Example 1, and were passaged one time. Media from each was assayed for the presence of G-CSF by ELISA. The approximate concentration of G-CSF in the media from cells transduced with p407-G-CSF-SIN was 40 ng/ml. The approximate concentration of G-CSF in the media from cells transduced with the Tombak vector was 249 ng/ml showing the potential for use of pTombak-hG-CSF in a transgenic avian system. In particular, the vector is expected to be particularly useful for producing exogenous protein in a transgenic avians (e.g., in the avian oviduct for packaging into eggs) produced as disclosed herein and produced as is know to practitioners of skill in the art.

What is claimed is:

1. A hybrid promoter comprising an LTR component and a promoter component that is 3′ of LTR component.

2. The hybrid promoter of claim 1 wherein the 3′ end of the LTR component is linked to the 5′ end of the promoter component.

3. The hybrid promoter of claim 1 wherein the hybrid promoter facilitates transcription of a coding sequence by an amount greater than that of the promoter component.

4. The hybrid promoter of claim 1 wherein the promoter is contained in a vector.

5. The hybrid promoter of claim 1 wherein the promoter is contained in a retroviral vector.

6. The hybrid promoter of claim 1 wherein the promoter is contained in a SIN retroviral vector.

7. The hybrid promoter of claim 1 wherein the promoter is contained in a retroviral vector and the LTR component of the hybrid promoter serves as the 5′ LTR of the vector.

8. The hybrid promoter of claim 1 wherein the LTR component comprises a portion of an LTR of a virus selected from the group consisting of Rous Sarcoma Virus (RSV), Murine Leukemia Virus (MLV), Molony Murine Sarcoma Virus (MMSV), Moloney Murine Leukemia Virus (MMLV), Avian Leokosis Virus (ALV) and lentivirus (e.g., human immunodeficiency virus (HIV), feline immunodeficiency virus (FIV), bovine immunodeficiency virus (BIV) and simian immunodeficiency virus (SIV).

9. The hybrid promoter of claim 1 wherein the promoter component comprises a portion of an promoter selected from the group consisting of CMV promoter, ef-1a promoter, PGK promoter and beta actin promoter.

10. The hybrid promoter of claim 1 wherein the promoter is operably linked to a nucleotide coding sequence.

11. The hybrid promoter of claim 10 wherein the nucleotide coding sequence encodes a therapeutic protein.

12. A transgenic avian containing a hybrid promoter comprising an LTR component and a promoter component.

13. The transgenic avian of claim 12 comprising a tubular gland cell containing the hybrid promoter.

14. The transgenic avian of claim 12 wherein the avian is selected from the group consisting of chicken, turkey, duck, goose, quail, pheasants, parrots, finches, hawks, crows and ratites including ostrich, emu and cassowary.

15. The transgenic avian of claim 12 wherein the 3′ end of the LTR component is linked to the 5′ end of the promoter component.

16. The transgenic avian of claim 12 wherein the hybrid promoter facilitates transcription of a coding sequence by an amount greater than that of the promoter component.

17. The transgenic avian of claim 12 wherein the promoter is contained in a vector.

18. The transgenic avian of claim 12 wherein the promoter is contained in a retroviral vector.

19. The transgenic avian of claim 12 wherein the promoter is contained in a SIN retroviral vector.

20. The transgenic avian of claim 12 wherein the promoter is contained in a retroviral vector and the LTR component of the hybrid promoter serves as the 5′ LTR of the vector.

21. The transgenic avian of claim 12 wherein the LTR component comprises a portion of an LTR of a virus selected from the group consisting of Rous Sarcoma Virus (RSV), Murine Leukemia Virus (MLV), Molony Murine Sarcoma Virus (MMSV), Moloney Murine Leukemia Virus (MMLV), Avian Leokosis Virus (ALV) and lentivirus (e.g., human immunodeficiency virus (HIV), feline immunodeficiency virus (FIV), bovine immunodeficiency virus (BIV) and simian immunodeficiency virus (SIV).

22. The transgenic avian of claim 12 wherein the promoter component comprises a portion of an promoter selected from the group consisting of CMV promoter, ef-1a promoter, PGK promoter and beta actin promoter.

23. The hybrid promoter of claim 12 wherein the promoter is operably linked to a nucleotide coding sequence.

24. A method comprising producing a protein comprising: producing a transgenic avian containing in its genome a hybrid promoter comprising an LTR component and a promoter component wherein the hybrid promoter is operably linked to a protein coding sequence and the protein is packaged into a hard shell egg laid by the avian.

25. The method of claim 24 wherein the protein is not normally produced in an avian.

26. The method of claim 24 wherein the protein is a therapeutic protein.


SEQUENCE LISTING (28 sequences)

SEQ ID NO: 1 - DNA, 3169 bp, Artificial Sequence
  Fragment of pNLB-CMV-Des-Arg166-EPO, synthetic expression vector
aatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgacgactacgagcacatgcatgaagcagaaggcttcatttggtgaccccgacgtgatcgttagggaatagtggtcggccacaggcggcgtggcgatcctgtcctcatccgtctcgcttattcggggagcggacgatgaccctagtagagggggctgcggcttaggagggcagaagctgagtggcgtcggagggagccctactgcagggggccaacataccctaccgagaactcagagagtcgttggaagacgggaaggaagcccgacgactgagcggtccaccccaggcgtgattccggttgctctgcgtgattccggtcgcccggtggatcaagcatggaagccgtcataaaggtgatttcgtccgcgtgtaagacctattgcgggaaaacctctccttctaagaaggaaataggggctatgttgtccctgttacaaaaggaagggttgcttacgtccccctcagacttatattccccggggtcctgggatcccattaccgcggcgctctctcagcgggctatggtacttggaaaatcgggagagttaaaaacctggggattggttttgggggcattgaaggcggctcgagggaagatcccatatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcaacagcgtgccgcagatcctctagaggatcgatccccctttgtatagtccatcctacaagagcaaaaacttgtaagcatttcaggtagcaaagtaatgaaaccacaaaatgctgaaaatgctccacagttgaatgcacagatatttatgttctgtgtttattcctgaagtgtaaggaatgtaaggaaatcatagattagttgtttctttcctttttgcagggcccagaccaccataagcttgggatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaccca

SEQ ID NO: 2 - DNA, 4975 bp, Artificial Sequence
  pNLB-407P-hG-CSF, synthetic expression vector
aatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatttggtgaccccgacgtgatcgttagggaatagtggtcggccacaggcggcgtggcgatcctgtcctcatccgtctcgcttattcggggagcggacgatgaccctagtagagggggctgcggcttaggagggcagaagctgagtggcgtcggagggagccctactgcagggggccaacataccctaccgagaactcagagagtcgttggaagacgggaaggaagcccgacgactgagcggtccaccccaggcgtgattccggttgctctgcgtgattccggtcgcccggtggatcaagcatggaagccgtcataaaggtgatttcgtccgcgtgtaagacctattgcgggaaaacctctccttctaagaaggaaataggggctatgttgtccctgttacaaaaggaagggttgcttacgtccccctcagacttatattccccggggtcctgggatcccattaccgcggcgctctctcagcgggctatggtacttggaaaatcgggagagttaaaaacctggggattggttttgggggcattgaaggcggctcgagggaagatcccatatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcaacagcgtgccgcagatcctctagaggatcgatccccctttgtatagtccatcctacaagagcaaaaacttgtaagcatttcaggtagcaaagtaatgaaaccacaaaatgctgaaaatgctccacagttgaatgcacagatatttatgttctgtgtttattcctgaagtgtaaggaatgtaaggaaatcatagattagttgtttctttcctttttgcagggcccagaccaccataagctacgcgtaatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcagcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagctttcaccatggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctagttgcctcccatctgcagagcttcctggaggtgtcgtaccgcgttctacgccaccttgcccagccctgaaaagtctagtatggggattggtggcgacgactcctggagcccgtcagtatcggcggaattccagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggcggatcccccgggctgcagaaccgagcggctattgacttcttgctcctagctcacggccatggctgtgaggacattgccggaatgtgttgtttcaatctgagtgatcacagtgagtctatacagaagaagttccagctaatgaaggaacatgtcaataagatcggcgtgaacaacgacccaatcggaagttggctgcgaggattattcggaggaataggagaatgggccgtacacttgctgaaaggactgcttttggggcttgtagttatcttgttgctagtagtatgcttgccttgccttttgcaatgtgtatctagtagtattcgaaagatgattgataattcactcggctatcgcgaggaatataaaaaaattacaggaggcttataagcagcccgaaagaagagcgtaggcgagttcttgtattccgtgtgatagctggttggattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggaaatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatt

SEQ ID NO: 3 - DNA, 2644 bp, Artificial Sequence
  Transgene Containing 407-Promoter
aatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcagcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctttcaccatgggcgtgcacgagtgccctgcttggctgtggctgctcttgagcctgctcagcctgcctctgggcctgcctgtgctgggcgctcctccaaggctgatctgcgatagcagggtgctggagaggtacctgctggaggctaaggaggctgagaacatcaccaccggctgcgctgagcactgcagcctgaacgagaacatcaccgtgcctgataccaaggtgaacttttacgcttggaagaggatggaggtgggccagcaggctgtggaggtgtggcagggcctggctctgctgagcgaggctgtgctgaggggccaggctctgctggtgaacagctctcagccttgggagcctctgcagctgcacgtggataaggctgtgagcggcctgagaagcctgaccaccctgctgagggctctgggcgctcagaaggaggctatcagccctccagatgctgcaagcgctgcccctctgaggaccatcaccgctgatacctttaggaagctgtttagggtgtacagcaactttctgaggggcaagctgaagctgtacaccggcgaggcttgcaggaccggcgattagaaagtctagtatggggattggtggcgacgactcctggagcccgtcagtatcggcggaattccagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggcggatcccccgggctgcagaaccgagcggctattgacttcttgctcctagctcacggccatggctgtgaggacattgccggaatgtgttgtttcaatctgagtgatcacagtgagtctatacagaagaagttccagctaatgaaggaacatgtcaataagatcggcgtgaacaacgacccaatcggaagttggctgcgaggattattcggaggaataggagaatgggccgtacacttgctgaaaggactgcttttggggcttgtagttatcttgttgctagtagtatgcttgccttgccttttgcaatgtgtatctagtagtattcgaaagatgattgataattcactcggctatcgcgaggaatataaaaaaattacaggaggcttataagcagcccgaaagaagagcgtaggcgagttcttgtattccgtgtgatagctggttggattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggaaatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgacgactacgagcacatgcatgaagcagaaggcttcatt

SEQ ID NO: 4 - DNA, 1888 bp, Artificial Sequence
  RSV LTR/Chicken Beta Actin promoter
aatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcattcaccggtgttattgctgctcggtgcgtgcatgcacatcagtgtcgctgcagctcagtgcatgcacgctcattgcccatcgctatccctgcctctcctgctggcgctccccgggaggtgacttcaaggggaccgcaggaccacctcgggggtggggggagggctgcacacgcggaccccgctccccctccccaacaaagcactgtggaatcaaaaaggggggaggggggatggaggggcgcgtcacacccccgccccacaccctcacctcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggggtttatatcttcccttctctgttcctccgcagccagcc

SEQ ID NO: 5 - DNA, 619 bp, Artificial Sequence
  RSV LTR/SV40 promoter
aatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcattctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgcctggggagcctggggactttccacaccctaactgacacacattccacagaa

SEQ ID NO: 6 - DNA, 859 bp, Artificial Sequence
  RSV LTR/PGK promoter
aatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttggctccttcgctttctgggctcagaggctgggaaggggtgggtccgggggcgggctcaggggcgggctcaggggcggggcgggcgcccgaaggtcctccggaagcccggcattctgcacgcttcaaaagcgcacgtctgccgcgctgttctcctcttcctcatctccgggcctttcgacctgca

SEQ ID NO: 7 - DNA, 1667 bp, Artificial Sequence
  RSV LTR/Mouse EF1 alpha promoter
aatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcattcactagtggagccgagagtaattcatacaaaaggagggatcgccttcgcaaggggagagcccagggaccgtccctaaattctcacagacccaaatccctgtagccgccccacgacagcgcgaggagcatgcgctcagggctgagcgcggggagagcagagcacacaagctcatagaccctggtcgtgggggggaggaccggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaa

SEQ ID NO: 8 - DNA, 1018 bp, Artificial Sequence
  HIV LTR/CMV promoter
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccgga

SEQ ID NO: 9 - DNA, 1877 bp, Artificial Sequence
  HIV LTR/Chicken beta actin promoter
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagcaccggtgttattgctgctcggtgcgtgcatgcacatcagtgtcgctgcagctcagtgcatgcacgctcattgcccatcgctatccctgcctctcctgctggcgctccccgggaggtgacttcaaggggaccgcaggaccacctcgggggtggggggagggctgcacacgcggaccccgctccccctccccaacaaagcactgtggaatcaaaaaggggggaggggggatggaggggcgcgtcacacccccgccccacaccctcacctcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggggtttatatcttcccttctctgttcctccgcagccagcc

SEQ ID NO: 10 - DNA, 608 bp, Artificial Sequence
  HIV LTR/SV40 promoter
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgcctggggagcctggggactttccacaccctaactgacacacattccacagaa

SEQ ID NO: 11 - DNA, 848 bp, Artificial Sequence
  HIV LTR/PGK promoter
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttggctccttcgctttctgggctcagaggctgggaaggggtgggtccgggggcgggctcaggggcgggctcaggggcggggcgggcgcccgaaggtcctccggaagcccggcattctgcacgcttcaaaagcgcacgtctgccgcgctgttctcctcttcctcatctccgggcctttcgacctgca

SEQ ID NO: 12 - DNA, 1656 bp, Artificial Sequence
  HIV LTR/mouse EF1 alpha promoter
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagcactagtggagccgagagtaattcatacaaaaggagggatcgccttcgcaaggggagagcccagggaccgtccctaaattctcacagacccaaatccctgtagccgccccacgacagcgcgaggagcatgcgctcagggctgagcgcggggagagcagagcacacaagctcatagaccctggtcgtgggggggaggaccggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaa

SEQ ID NO: 13 - DNA, 1272 bp, Artificial Sequence
  MMLV LTR/CMV promoter
ttttgaaagaccccacccgtaggtggcaagctagcttaagtaacgccactttgcaaggcatggaaaaatacataactgagaatagaaaagttcagatcaaggtcaggaacaaagaaacagctgaataccaaacaggatatctgtggtaagcggttcctgccccggctcagggccaagaacagatgagacagctgagtgatgggccaaacaggatatctgtggtaagcagttcctgccccggctcggggccaagaacagatggtccccagatgcggtccagccctcagcagtttctagtgaatcatcagatgtttccagggtgccccaaggacctgaaaatgaccctgtaccttatttgaactaaccaatcagttcgcttctcgcttctgttcgcgcgcttccgctctccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccgga

SEQ ID NO: 14 - DNA, 2131 bp, Artificial Sequence
  MMLV LTR/chicken beta actin promoter
ttttgaaagaccccacccgtaggtggcaagctagcttaagtaacgccactttgcaaggcatggaaaaatacataactgagaatagaaaagttcagatcaaggtcaggaacaaagaaacagctgaataccaaacaggatatctgtggtaagcggttcctgccccggctcagggccaagaacagatgagacagctgagtgatgggccaaacaggatatctgtggtaagcagttcctgccccggctcggggccaagaacagatggtccccagatgcggtccagccctcagcagtttctagtgaatcatcagatgtttccagggtgccccaaggacctgaaaatgaccctgtaccttatttgaactaaccaatcagttcgcttctcgcttctgttcgcgcgcttccgctctccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatcaccggtgttattgctgctcggtgcgtgcatgcacatcagtgtcgctgcagctcagtgcatgcacgctcattgcccatcgctatccctgcctctcctgctggcgctccccgggaggtgacttcaaggggaccgcaggaccacctcgggggtggggggagggctgcacacgcggaccccgctccccctccccaacaaagcactgtggaatcaaaaaggggggaggggggatggaggggcgcgtcacacccccgccccacaccctcacctcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggggtttatatcttcccttctctgttcctccgcagccagcc

SEQ ID NO: 15 - DNA, 862 bp, Artificial Sequence
  MMLV LTR/SV40 promoter
ttttgaaagaccccacccgtaggtggcaagctagcttaagtaacgccactttgcaaggcatggaaaaatacataactgagaatagaaaagttcagatcaaggtcaggaacaaagaaacagctgaataccaaacaggatatctgtggtaagcggttcctgccccggctcagggccaagaacagatgagacagctgagtgatgggccaaacaggatatctgtggtaagcagttcctgccccggctcggggccaagaacagatggtccccagatgcggtccagccctcagcagtttctagtgaatcatcagatgtttccagggtgccccaaggacctgaaaatgaccctgtaccttatttgaactaaccaatcagttcgcttctcgcttctgttcgcgcgcttccgctctccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgcctggggagcctggggactttccacaccctaactgacacacattccacagaa

SEQ ID NO: 16 - DNA, 1102 bp, Artificial Sequence
  MMLV LTR/PGK promoter
ttttgaaagaccccacccgtaggtggcaagctagcttaagtaacgccactttgcaaggcatggaaaaatacataactgagaatagaaaagttcagatcaaggtcaggaacaaagaaacagctgaataccaaacaggatatctgtggtaagcggttcctgccccggctcagggccaagaacagatgagacagctgagtgatgggccaaacaggatatctgtggtaagcagttcctgccccggctcggggccaagaacagatggtccccagatgcggtccagccctcagcagtttctagtgaatcatcagatgtttccagggtgccccaaggacctgaaaatgaccctgtaccttatttgaactaaccaatcagttcgcttctcgcttctgttcgcgcgcttccgctctccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttggctccttcgctttctgggctcagaggctgggaaggggtgggtccgggggcgggctcaggggcgggctcaggggcggggcgggcgcccgaaggtcctccggaagcccggcattctgcacgcttcaaaagcgcacgtctgccgcgctgttctcctcttcctcatctccgggcctttcgacctgca

SEQ ID NO: 17 - DNA, 1910 bp, Artificial Sequence
  MMLV LTR/mouse EF1 alpha promoter
ttttgaaagaccccacccgtaggtggcaagctagcttaagtaacgccactttgcaaggcatggaaaaatacataactgagaatagaaaagttcagatcaaggtcaggaacaaagaaacagctgaataccaaacaggatatctgtggtaagcggttcctgccccggctcagggccaagaacagatgagacagctgagtgatgggccaaacaggatatctgtggtaagcagttcctgccccggctcggggccaagaacagatggtccccagatgcggtccagccctcagcagtttctagtgaatcatcagatgtttccagggtgccccaaggacctgaaaatgaccctgtaccttatttgaactaaccaatcagttcgcttctcgcttctgttcgcgcgcttccgctctccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatcactagtggagccgagagtaattcatacaaaaggagggatcgccttcgcaaggggagagcccagggaccgtccctaaattctcacagacccaaatccctgtagccgccccacgacagcgcgaggagcatgcgctcagggctgagcgcggggagagcagagcacacaagctcatagaccctggtcgtgggggggaggaccggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaa

SEQ ID NO: 18 - DNA, 6219 bp, Artificial Sequence
  pSIN-407-hG-CSF, synthetic expression vector
aattgctagactaggatcccccgtgctgcagaaccgagcggctattgacttcttgctcctagctcacggccatggctgtgaggacattgcgggaatgtgttgtttcaatctgagtgatcacagtgagtctatacagaagaagttccagctaatgaaggaacatgtcaataagatcggcgtgaacaacgacccaatcggaagttggctgcgaggattattcggaggaataggagaatgggccgtacacttgctgaaaggactgcttttggggcttgtagttatcttgttgctagtagtatgcttgccttgccttttgcaatgtgtatctagtagtattcgaaagatgattgataattcactcggctatcgcgaggaatataaaaaaattacaggaggcttataagcagcccgaaagaagagcgtaggcgagttcttgtattccgtgtgatagctggttggattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatttggtgaccccgacgtgatcgttagggaatacgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggaaatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatttggtgaccccgacgtgatcgttagggaatagtggtcggccacaggcggcgtggcgatcctgtcctcatccgtctcgcttattcggggagcggacgatgaccctagtagagggggctgcggcttaggagggcagaagctgagtggcgtcggagggagccctactgcagggggccaacataccctaccgagaactcagagagtcgttggaagacgggaaggaagcccgacgactgagcggtccaccccaggcgtgattccggttgctctgcgtgattccggtcgcccggtggatcaagcatggaagccgtcataaaggtgatttcgtccgcgtgtaagacctattgcgggaaaacctctccttctaagaaggaaataggggctatgttgtccctgttacaaaaggaagggttgcttacgtccccctcagacttatattccccggggtcctgggatcccattaccgcggcgctctctcagcgggctatggtacttggaaaatcgggagagttaaaaacctggggattggttttgggggcattgaaggcggctcgacgcgtaatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcagcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagctttcaccatggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctagttgcctcccatctgcagagcttcctggaggtgtcgtaccgcgttctacgccaccttgcccagccctgaaaagtctagtatggggattggtggcgacgactcctggagcccgtcagtatcggcggaattccagctga

SEQ ID NO: 19 - DNA, 6411 bp, Artificial Sequence
  pTombak-G-CSF, synthetic expression vector
cagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctagttgcctcccatctgcagagcttcctggaggtgtcgtaccgcgttctacgccaccttgcccagccctgaaaagtctagtatggggattggtggcgacgactcctggagcccgtcagtatcggcggaattccagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggcggatcccccgggctgcagaaccgagcggctattgacttcttgctcctagctcacggccatggctgtgaggacattgccggaatgtgttgtttcaatctgagtgatcacagtgagtctatacagaagaagttccagctaatgaaggaacatgtcaataagatcggcgtgaacaacgacccaatcggaagttggctgcgaggattattcggaggaataggagaatgggccgtacacttgctgaaaggactgcttttggggcttgtagttatcttgttgctagtagtatgcttgccttgccttttgcaatgtgtatctagtagtattcgaaagatgattgataattcactcggctatcgcgaggaatataaaaaaattacaggaggcttataagcagcccgaaagaagagcgtaggcgagttcttgtattccgtgtgatagctggttggattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggaaatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatttggtgaccccgacgtgatcgttagggaatagtggtcggccacaggcggcgtggcgatcctgtcctcatccgtctcgcttattcggggagcggacgatgaccctagtagagggggctgcggcttaggagggcagaagctgagtggcgtcggagggagccctactgcagggggccaacataccctaccgagaactcagagagtcgttggaagacgggaaggaagcccgacgactgagcggtccaccccaggcgtgattccggttgctctgcgtgattccggtcgcccggtggatcaagcatggaagccgtcataaaggtgatttcgtccgcgtgtaagacctattgcgggaaaacctctccttctaagaaggaaataggggctatgttgtccctgttacaaaaggaagggttgcttacgtccccctcagacttatattccccggggtcctgggatcccattaccgcggcgctctctcagcgggctatggtacttggaaaatcgggagagttaaaaacctggggattggttttgggggcattgaaggcggctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgcaatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcagcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggatggtgaccccgacgtgatcgttagggaatagtggtcggccacaggcggcgtggcgatcctgtcctcatccgtctcgcttattcggggagcggacgatgaccctagtagagggggctgcggcttaggagggcagaagctgagtggcgtcggagggagccctactgcagggggccaacataccctaccgagaactcagagagtcgttggaagacgggaaggaagcccgacgactgagcggtccaccccaggcgtgattccggttgctctgcgtgattccggtcgcccggtggatcaagcatggctggacctgccacc

SEQ ID NO: 20 - DNA, 18 bp, Artificial Sequence
  SJ-EPO-for, primer
gccctccagatgctgcaa

SEQ ID NO: 21 - DNA, 26 bp, Artificial Sequence
  SJ-EPO-rev, primer
ccctaaacagcttcctaaaggtatca

SEQ ID NO: 22 - DNA, 22 bp, Artificial Sequence
  Taqman EPO probe
cgctgcccctctgaggaccatc

SEQ ID NO: 23 - DNA, 49 bp, Artificial Sequence
  407-pro-for, primer
ggcgtctcaagctacgcgtaatgtagtcttatgcaatactcttgtagtc

SEQ ID NO: 24 - DNA, 32 bp, Artificial Sequence
  407-pro-rev, primer
cgcccatggtgaaagcttccggtctccctata

SEQ ID NO: 25 - DNA, 24 bp, Artificial Sequence
  SJ-G-CSF for, primer
cagagcttcctgctcaagtgctta

SEQ ID NO: 26 - DNA, 22 bp, Artificial Sequence
  SJ-G-CSF rev, primer
ttgtaggtggcacacagcttct

SEQ ID NO: 27 - DNA, 24 bp, Artificial Sequence
  SJ-G-CSF probe
agcaagtgaggaagatccagggcg

SEQ ID NO: 28 - DNA, 6722 bp, Artificial Sequence
  pAVIJCR-A395.22.3.1-KM, synthetic expression vector
ctagactaggatcccccgtgctgcagaaccgagcggctattgacttcttgctcctagctcacggccatggctgtgaggacattgcgggaatgtgttgtttcaatctgagtgatcacagtgagtctatacagaagaagttccagctaatgaaggaacatgtcaataagatcggcgtgaacaacgacccaatcggaagttggctgcgaggattattcggaggaataggagaatgggccgtacacttgctgaaaggactgcttttggggcttgtagttatcttgttgctagtagtatgcttgccttgccttttgcaatgtgtatctagtagtattcgaaagatgattgataattcactcggctatcgcgaggaatataaaaaaattacaggaggcttataagcagcccgaaagaagagcgtaggcgagttcttgtattccgtgtgatagctggttggattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatttggtgaccccgacgtgatcgttagggaatacgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcattggtaattgatcggctggcacgcggaatataggaggtcgctgaatagtaaacttgtagacttggctacagcatagagtatcttctgtagctctgatgactgctaggaaataatgctacggataatgtggggagggcaaggcttgcgaatcgggttgtaacgggcaaggcttgactgaggggacaatagcatgtttaggcgaaaagcggggcttcggttgtacgcggttaggagtcccctcaggatatagtagtttcgcttttgcatagggagggggaaatgtagtcttatgcaatactcttgtagtcttgcaacatgcttatgtaacgatgagttagcaacatgccttataaggagagaaaaagcaccgtgcatgccgattggtgggagtaaggtggtatgatcgtggtatgatcgtgccttgttaggaaggcaacagacgggtctaacacggattggacgaaccactgaattccgcattgcagagatattgtatttaagtgcctagctcgatacaataaacgccatttgaccattcaccacattggtgtgcacctgggttgatggccggaccgttgattccctgrcgactacgagcacatgcatgaagcagaaggcttcatttggtgaccccgacgtgatcgttagggaatagtggtcggccacaggcggcgtggcgatcctgtcctcatccgtctcgcttattcggggagcggacgatgaccctagtagagggggctgcggcttaggagggcagaagctgagtggcgtcggagggagccctactgcagggggccaacataccctaccgagaactcagagagtcgttggaagacgggaaggaagcccgacgactgagcggtccaccccaggcgtgattccggttgctctgcgtgattccggtcgcccggtggatcaagcatggaagccgtcataaaggtgatttcgtccgcgtgtaagacctattgcgggaaaacctctccttctaagaaggaaataggggctatgttgtccctgttacaaaaggaagggttgcttacgtccccctcagacttatattccccggggtcctgggatcccattaccgcggcgctctctcagcgggctatggtacttggaaaatcgggagagttaaaaacctggggattggttttgggggcattgaaggcggctcgagatctgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccggcgctagatcccgattacttcaagagagtaatcgggatctagcgccttttttgctagcgaattcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagctttcaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagaaagtctagggtaccaattg